SEQUENCE LISTING (6 sequences)

SEQ ID NO: 1 - DNA, 20 bp, Artificial Sequence
  Primer
cctgacctacggcaacagat

SEQ ID NO: 2 - DNA, 20 bp, Artificial Sequence
  Primer
ctcttcatcagctcgtctgc

SEQ ID NO: 3 - DNA, 21 bp, Artificial Sequence
  Primer
tctgcgggagccagatgcact

SEQ ID NO: 4 - DNA, 22 bp, Artificial Sequence
  Primer
gatgatcccttgctgacttacc

SEQ ID NO: 5 - DNA, 21 bp, Artificial Sequence
  Primer
gtcgtcctcaaccacagtcac

SEQ ID NO: 6 - DNA, 25 bp, Artificial Sequence
  Primer
ccactggggaagaagtggccatgcg

(19) United States
(12) Patent Application Publication
(10) Pub. No.: US 2008/0003575 A1
(43) Pub. Date: Jan. 3, 2008

(54) Methods and composition for RNA extraction

(75) Inventors: Steve S. Michalik, St. Louis, MO (US); Scott A. Weber, St. Louis, MO (US); Carol A. Kreader, St. Louis, MO (US)

Correspondence Address: SENNIGER POWERS (SGM), ONE METROPOLITAN SQUARE, 16TH FLOOR, ST. LOUIS, MO, 63102, US

(73) Assignee: Sigma-Aldrich Co., St. Louis, MO (US)

(21) Appl. No.: 11/476,273

(22) Filed: Jun. 28, 2006

(51) Int. Cl. C12Q 1/68; C07H 21/02

(52) U.S. Cl. 435/6; 536/25.4

(57) ABSTRACT

The present invention provides methods and compositions for extracting RNA from cells. The cellular extract may be directly used in a variety of reactions, such as reverse transcription and PCR.

### BACKGROUND OF THE INVENTION

The present invention provides methods and compositions for extracting RNA from cells. Also provided are methods and compositions for performing reverse transcription and PCR with these extracts.

The ability to study nucleic acids in biological samples has been important in biological and biochemical research. Reverse transcription followed by the quantitative polymerase chain reaction (qRT-PCR) is one of the main methods used for measuring mRNA levels from a small number of cells. RT-PCR is useful for detecting RNA species such as in quantitative analysis of gene expression, validation of mRNA knockdown by siRNA, signal amplification in in-situ hybridizations, as well as for other applications.

The application of RT-PCR and other methods in molecular biology require the extraction of RNA from biological samples. A number of approaches have been devised for performing such extractions. These approaches have included treating or manipulating cells in order to lyse the cells and release RNA, along with other cellular components. Some techniques have lysed cells using enzymatic activity, for example, by treating the cells with an enzyme such as proteinase K. Other techniques have treated cells with chaotropes and/or detergents or have used freeze thawing or snap freeze techniques to lyse cells.

One drawback of lysing cells using these techniques is that the resulting crude lysate typically contains not only RNA, but also a large amount of other cellular components. For example, most cells contain some type of RNase that may contribute to RNA degradation. High concentrations of RNase activity in the crude lysate may make it more difficult to maintain the integrity of the RNA in the lysate. Furthermore, the crude lysate may contain DNA, which may interfere with RT-PCR. Reagents added to lyse cells may also interfere with RT-PCR. Consequently, it is usually necessary to purify RNA from the crude lysate prior to use in RT-PCR reactions. RNA purification often includes organic extraction or silica binding, which require centrifugation or vacuum filtration. In some instances, it may be necessary to treat the crude lysate with enzymes capable of degrading or inactivating contaminating cellular debris.

Furthermore, many of the known techniques for extracting RNA from cells are labor intensive, often requiring specialized equipment and/or numerous steps. For example, enzymatic lysis often requires a heating and/or incubation step to inactivate the enzymes prior to performing RT-PCR or other such reactions. Furthermore, techniques such as freeze thawing or snap freezing may require specialized conditions and equipment in order to perform the lysis.

### SUMMARY OF THE INVENTION

Among the various aspects of the invention is the provision of a method for extracting RNA from cells. Advantageously, the method does not require the use of enzymes or any specialized equipment such as a centrifuge, vacuum or pressure system, or freezing or heating devices. The cellular extract produced using the methods of the present invention may be directly used in reverse transcription or RT-PCR reactions without first purifying or isolating RNA from other cellular debris.

One aspect of the present invention is a method for extracting RNA from cells. The method comprises combining a cell population with an extraction medium to form a cellular extract containing extracted RNA, a salt selected from the group consisting of monovalent salts, divalent salts, and combinations thereof, and a detergent selected from the group consisting of non-ionic detergents, zwitterionic detergents, and combinations thereof. The concentration of the detergent in the cellular extract is about 0.1% to about 10% by weight, and the concentration of the salt in the cellular extract is about 10 mM to about 5 M.

Another aspect of the present invention is a method for preparing cDNA. The method comprises combining a cell population with an extraction medium to form a cellular extract containing extracted RNA, a salt selected from the group consisting of monovalent salts, divalent salts, and combinations thereof, and a detergent selected from the group consisting of non-ionic detergents, zwitterionic detergents, and combinations thereof, the concentration of the detergent in the cellular extract being about 0.1% to about 10% by weight and the concentration of the salt in the cellular extract being about 10 mM to about 5 M; combining the cellular extract with a reverse transcriptase to form a first reaction mixture; and incubating the first reaction mixture to produce a cDNA.

Other objects and features will be in part apparent and in part pointed out hereinafter.

### BRIEF DESCRIPTION OF THE DRAWINGS

FIGS. 1A and 1B show charts depicting the average Ct values for GAPDH (FIG. 1A) or PGK1 (FIG. 1 B) obtained in qRT-PCR with cellular extracts or purified RNA prepared with different extraction solutions or commercially available kits, as discussed in Example 1.

FIGS. 2A, 2B, 2C, and 2D show charts depicting Ct values for GAPDH or PGK1 with cellular extracts prepared using either Ambion (Austin, Tex.) Cells-to-Signal kit, or extraction solution B or E either with or without an RNase inhibitor, as discussed in Example 2. FIG. 2A depicts GAPDH Ct values for extracts prepared without RNase inhibitor; FIG. 2B depicts GAPDH Ct values for extracts prepared with RNase inhibitor; FIG. 2C depicts PGK1 Ct values for extracts prepared without RNase inhibitor; and FIG. 2D depicts PGK1 Ct values for extracts prepared with RNase inhibitor.

FIG. 3 shows a chart depicting G6PD and LMNA Ct values for reaction mixtures in which 50 mM KCl was either added or omitted from the reaction mixture, as discussed in Example 4.

FIG. 4 shows a chart depicting a plot of PGK1 Ct values vs. number of Panc 1 cells per well for cellular extracts prepared using different amounts of an extraction solution, as discussed in Example 5.

FIGS. 5A and 5B show charts depicting Ct values from one-step (FIG. 5A) or two-step (FIG. 5B) qRT-PCR performed using extracts of cells transfected with siRNA targeting a specific target (designated “siRNA”) or extracts of cells prepared using siControl (designated “non-target”) and assays for the target mRNAs, as discussed in Example 6. FIG. 5C shows a chart comparing the percent knockdown for one-step and two-step RT-PCR for each siRNA target, as discussed in Example 6.

FIGS. 6A and 6B show charts comparing the Ct values from two-step qRT-PCR performed using either probe-based (FIG. 6A) or SYBR-based (FIG. 6B) specific gene expression assays and RNA obtained using extraction solution E (designated “X”) or a commercially available kit (designated “Q”), as discussed in Example 7.

### DETAILED DESCRIPTION OF THE INVENTION

The present invention is directed to methods, compositions, and kits for extracting RNA from cells. The cellular extract may be directly used in a variety of reactions, such as reverse transcription and PCR.

### Cell Populations

The present invention provides methods and kits for extracting RNA from cell populations using an extraction medium that comprises a salt and a detergent. The cell populations may comprise any cell or virus that comprises RNA (e.g., mRNA, tRNA, rRNA, or non-coding RNA). The cell may be any of a variety of different types of cells including, for example, eukaryotic cells such as fungal, protist, plant, or animal cells. Preferably the cell is a mammalian cell, such as a rodent, mouse, rat, hamster, primate, or human cell. The cells may be living, dead, or damaged, that is, having disruptions in the cell wall or cell membrane. The cell may be obtained from any source, as will be understood by those of skill in the art, including from a cell culture, from a sample collected from a subject (e.g., from an animal including a human) or the environment, from a tissue sample or body fluid (e.g., whole blood, plasma, serum, urine, or cerebral spinal fluid), and other such sources.

The cell population may be directly contacted with the extraction medium, or alternately, the cell population may be first concentrated by methods such as centrifugation, binding to a surface through immunoadsorption or other interaction, or filtration, prior to contact with the extraction medium. Optionally, the number of cells in the population may be increased by growing the cells on culture plates or in a suitable liquid medium prior to concentration or direct extraction. Methods and media for growing cells are well known to those of skill in the art.

In one exemplary embodiment, the cell population is prepared by growing the cells in a suitable medium, harvesting the cells, and optionally washing the cells to remove contaminants prior to contacting the cells with the extraction medium. For example, for cells in a cell suspension, the cells may be harvested from the growth medium by or by centrifugation at a force of from about 1 to about 100,000 xg, more preferably at a force of from about 100 to about 1,0000 xg, and preferably about 300 xg for about 0.01 to about 1500 minutes, and preferably for about 5 minutes. The growth medium may be removed by any suitable method including, for example, aspiration. In one embodiment, the cell pellet may be washed using a suitable wash solution (e.g., PBS), repelleted as described above, and the wash solution removed by aspiration. The resulting cell population may then be contacted with the extraction medium, as described herein. A similar method may be used to remove the growth media from cells attached to a substrate (e.g., a tube or plate). In this instance, the cells can be contacted directly with extraction media after removal of growth media without the need for harvesting cells via trypsinization and centrifugation.

### Extraction Medium

Once a suitable cell population has been obtained, RNA may be extracted from the cells using an extraction medium. The extraction medium of the present invention causes the release of RNA from cells present in the sample. In one preferred embodiment, the extraction medium comprises a detergent, a salt, and optionally other components that aid in the extraction and/or in reverse transcription or PCR reactions. Without wishing to be bound by any particular theory, it is believed that the extraction medium ruptures the cells through the action of the detergent and the salt. The detergent aids in the extraction by perforating the cell membrane, while the salt renders the extraction medium hypertonic. Under these hypertonic conditions, RNA is released from the cytosol of the ruptured cells through osmotic pressure exerted on the cell wall and/or cell membrane as the cell collapses in on itself. Advantageously, under these conditions it is believed the genomic DNA (gDNA) remains associated with the nucleus and other cellular debris. Likewise, fewer RNases are available to digest the extracted RNA because the RNases also remain trapped in the cellular debris, or in some instances, may be present in the extract only at insignificant concentrations.

Typically, the detergents and their concentrations used in the extraction medium are selected so as not to interfere with any reactions in which the extract may be used, particularly reverse transcription or PCR. Preferably, the extraction medium comprises a non-ionic detergent and/or a zwitterionic detergent. Non-ionic detergents are particularly preferred for use in the extraction medium because unlike some other detergents commonly used to lyse cells, non-ionic detergents can perforate the cell membrane to allow release of the RNA without rupturing the cell and/or organelles to such an extent that large amounts of DNA and other cellular components are also released.

Examplary non-ionic detergents for use in the extraction medium include BigCHAP (i.e. N,N-bis[3-(D-gluconamido)propyl]cholamide); bis(polyethylene glycol bis[imidazoyl carbonyl]); polyoxyethylene alcohols, such as Brij® 30 (polyoxyethylene(4)lauryl ether), Brij® 35 (polyoxyethylene(23)lauryl ether), Brij® 35P, Brij® 52 (polyoxyethylene 2 cetyl ether), Brij® 56 (polyoxyethylene 10 cetyl ether), Brij® 58 (polyoxyethylene 20 cetyl ether), Brij® 72 (polyoxyethylene 2 stearyl ether), Brij® 76 (polyoxyethylene 10 stearyl ether), Brij® 78 (polyoxyethylene 20 stearyl ether), Brij® 78P, Brij® 92 (polyoxyethylene 2 oleyl ether); Brij® 92V (polyoxyethylene 2 oleyl ether), Brij® 96V, Brij® 97 (polyoxyethylene 10 oleyl ether), Brij® 98 (polyoxyethylene(20)oleyl ether), Brij® 58P, and Brij® 700 (polyoxyethylene(100)stearyl ether); Cremophor® EL (i.e. polyoxyethylenglyceroltriricinoleat 35; polyoxyl 35 castor oil); decaethylene glycol monododecyl ether; decaethylene glycol mono hexadecyl ether; decaethylene glycol mono tridecyl ether; N-decanoyl-N-methylglucamine; n-decyl α-D-glucopyranoside; decyl β-D-maltopyranoside; digitonin; n-dodecanoyl-N-methylglucamide; n-dodecyl α-D-maltoside; n-dodecyl β-D-maltoside; heptaethylene glycol monodecyl ether; heptaethylene glycol monododecyl ether; heptaethylene glycol monotetradecyl ether; n-hexadecyl β-D-maltoside;hexaethylene glycol monododecyl ether; hexaethylene glycol monohexadecyl ether; hexaethylene glycol monooctadecyl ether; hexaethylene glycol monotetradecyl ether; Igepal® CA-630 (i.e. nonylphenyl-polyethylenglykol, (octylphenoxy)polyethoxyethanol, octylphenyl-polyethylene glycol); methyl-6-O-(N-heptylcarbamoyl)-α-D-glucopyranoside; nonaethylene glycol monododecyl ether; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-D-glucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether W-1; polyoxyethylene 10 tridecyl ether; polyoxyethylene 100 stearate; polyoxyethylene 20 isohexadecyl ether; polyoxyethylene 20 oleyl ether; polyoxyethylene 40 stearate; polyoxyethylene 50 stearate; polyoxyethylene 8 stearate; polyoxyethylene bis(imidazolyl carbonyl); polyoxyethylene 25 propylene glycol stearate; saponin from quillaja bark; sorbitan fatty acid esters, such as Span® 20 (sorbitan monolaurate), Span® 40 (sorbitane monopalmitate), Span® 60 (sorbitane monostearate), Span® 65 (sorbitane tristearate), Span® 80 (sorbitane monooleate), and Span® 85 (sorbitane trioleate); various alkyl ethers of polyethylene glycols, such as Tergitol® Type 15-S-12, Tergitol® Type 15-S-30, Tergitol® Type 15-S-5, Tergitol® Type 15-S-7, Tergitol® Type 15-S-9, Tergitol® Type NP-10 (nonylphenol ethoxylate), Tergitol® Type NP-4, Tergitol® Type NP-40, Tergitol® Type NP-7, Tergitol® Type NP-9 (nonylphenol polyethylene glycol ether), Tergitol® MIN FOAM 1x, Tergitol® MIN FOAM 2x, Tergitol®) Type TMN-10 (polyethylene glycol trimethylnonyl ether), Tergitol® Type TMN-6 (polyethylene glycol trimethylnonyl ether), Triton® 770, Triton® CF-10 (benzyl-polyethylene glycol tert-octylphenyl ether), Triton® CF-21, Triton® CF-32, Triton® DF-12, Triton® DF-16, Triton® GR-5M, Triton® N-42, Triton® N-57, Triton® N-60, Triton® N-101 (i.e. polyethylene glycol nonylphenyl ether; polyoxyethylene branched nonylphenyl ether), Triton® QS-15, Triton® QS-44, Triton® RW-75 (i.e. polyethylene glycol 260 mono(hexadecyl/octadecyl) ether and 1-octadecanol), Triton® SP-135, Triton® SP-190, Triton® W-30, Triton® X-15, Triton® X-45 (i.e. polyethylene glycol 4-tert-octylphenyl ether; 4-(1,1,3,3-tetramethylbutyl)phenyl-polyethylene glycol), Triton® X-100 (t-octylphenoxypolyethoxyethanol; polyethylene glycol tert-octylphenyl ether; 4-(1,1,3,3-tetramethylbutyl)phenyl-polyethylene glycol), Triton® X-102, Triton® X-114 (polyethylene glycol tert-octylphenyl ether; (1,1,3,3-tetramethylbutyl)phenyl-polyethylene glycol), Triton® X-165, Triton® X-305, Triton® X-405 (i.e. polyoxyethylene(40) isooctylcyclohexyl ether; polyethylene glycol tert-octylphenyl ether), Triton® X-705-70, Triton® X-151, Triton® X-200, Triton® X-207, Triton® X-301, Triton® XL-80N, and Triton® XQS-20; tetradecyl-β-D-maltoside; tetraethylene glycol monodecyl ether; tetraethylene glycol monododecyl ether; tetraethylene glycol monotetradecyl ether; triethylene glycol monodecyl ether; triethylene glycol monododecyl ether; triethylene glycol monohexadecyl ether; triethylene glycol monooctyl ether; triethylene glycol monotetradecyl ether; polyoxyethylene sorbitan fatty acid esters, such as TWEEN® 20 (polyethylene glycol sorbitan monolaurate), TWEEN® 20 (polyoxyethylene (20) sorbitan monolaurate), TWEEN® 21 (polyoxyethylene (4) sorbitan monolaurate), TWEEN® 40 (polyoxyethylene (20) sorbitan monopalmitate), TWEEN® 60 (polyethylene glycol sorbitan monostearate; polyoxyethylene (20) sorbitan monostearate), TWEEN® 61 (polyoxyethylene (4) sorbitan monostearate), TWEEN® 65 (polyoxyethylene (20) sorbitantristearate), TWEEN® 80 (polyethylene glycol sorbitan monooleate; polyoxyethylene (20) sorbitan monooleate), TWEEN® 81 (polyoxyethylene (5) sorbitan monooleate), and TWEEN® 85 (polyoxyethylene (20) sorbitan trioleate); tyloxapol; n-undecyl β-D-glucopyranoside, MEGA-8 (octanoyl-N-methylglucamide); MEGA-9 (nonanoyl-N-methylglucamide); MEGA-10 (decanoyl-N-methylglucamide); methylheptylcarbamoyl glucopyranoside; octyl-glucopyranoside; octyl-thioglucopyranoside; octyl-β-thioglucopyranoside; and various combinations thereof.

In one embodiment, the non-ionic detergent is selected from the group consisting of alkyl glucosides, alkyl maltosides, alkyl thioglucosides, BigCHAP series detergents, glucamides, polyoxyethylenes, and combinations thereof. Exemplary alkyl glucosides include n-decyl α-D-glucopyranoside, methyl-6-O-(N-heptylcarbamoyl)-α-D-glucopyranoside, n-undecyl β-D-glucopyranoside, methylheptylcarbamoyl glucopyranoside, octyl-glucopyranoside, and octyl-β-D-glucopyranoside. Exemplary alkyl maltosides include n-dodecyl α-D-maltoside, n-dodecyl β-D-maltoside, n-hexadecyl β-D-maltoside, tetradecyl-β-D-maltoside, and decyl β-D-maltopyranoside. Exemplary alkyl thioglucosides include octyl-β-thioglucopyranoside and octyl-thioglucopyranoside. Exemplary glucamides include n-dodecanoyl-N-methylglucamide, MEGA-8, MEGA-9, and MEGA-10. Exemplary polyoxyethylenes include polyoxyethylene alcohols, such as Brij® 30, Brij® 35, Brij® 35P, Brij® 52, Brij® 56, Brij® 58, Brij® 72, Brij® 76, Brij® 78, Brij® 78P, Brij® 92; Brij® 92V, Brij® 96V, Brij® 97, Brij® 98, Brij® 58P, and Brij® 700, Cremophor® EL, polyoxyethylene 10 tridecyl ether, polyoxyethylene 100 stearate, polyoxyethylene 20 isohexadecyl ether, polyoxyethylene 20 oleyl ether, polyoxyethylene 40 stearate, polyoxyethylene 50 stearate, polyoxyethylene 8 stearate, polyoxyethylene bis(imidazolyl carbonyl), polyoxyethylene 25 propylene glycol stearate, Triton® 770, Triton® CF-10, Triton® CF-21, Triton® CF-32, Triton® DF-12, Triton® DF-16, Triton® GR-5M, Triton® N-42, Triton® N-57, Triton® N-60, Triton® N-101, Triton® QS-15, Triton® QS-44, Triton® RW-75, Triton® SP-135, Triton® SP-190, Triton® W-30, Triton® X-15, Triton® X-45, Triton® X-100, Triton® X-1 02, Triton® X-114, Triton® X-165, Triton® X-305, Triton® X-405, Triton® X-705-70, Triton® X-151, Triton® X-200, Triton® X-207, Triton® X-301, Triton® XL-80N, and Triton® XQS-20, and polyoxyethylene sorbitan fatty acid esters, such as TWEEN® 20, TWEEN® 21, TWEEN® 40, TWEEN® 60, TWEEN® 61, TWEEN® 65, TWEEN® 80, TWEEN® 81, and TWEEN® 85. More preferably, the polyoxyethylene is Triton® X-100.

In certain embodiments, the extraction medium may comprise a zwitterionic detergent. Preferably, the zwitterionic detergent is one that is compatible for use in reverse transcription and/or RT-PCR reactions. Examples of such zwitterionic detergents include CHAPS (i.e. 3-[(3-cholamidopropyl)dimethylammonio]-1-propanesulfonate); CHAPSO (i.e. 3-[(3-cholamidopropyl)dimethylammonio]-2-hydroxy-1-propanesulfonate); N-dodecylmaltoside; α-dodecyl-maltoside; β-dodecyl-maltoside; 3-(decyldimethylammonio)propanesulfonate inner salt; 3-(N,N-(dodecyldimethylammonio)propanesulfonate inner salt; 3-(N,N-dimethylmyristylammonio)propanesulfonate; 3-(N,N-dimethyloctadecylammonio)propanesulfonate; 3-(N,N-dimethyloctylammonio)propanesulfonate inner salt; 3-(N,N-dimethylpalmitylammonio)propanesulfonate; and betaines, including sulfobetaines and carbobetaines. Sulfobetaines include, for example, SB3-8, SB3-10, SB3-12, SB3-14, SB3-16, and SB3-18. Preferably, the zwitterionic detergent is a betaine.

The extraction medium preferably comprises from about 0.1% to about 10% by weight of the detergent, more preferably from about 0.5% to about 5% by weight of the detergent, and still more preferably about 1% by weight of the detergent.

In addition to the detergent, the extraction medium further comprises a salt. As will be recognized by those skilled in the art, certain salts may interfere with or inhibit reverse transcription or PCR reactions. As such, the salt and the concentration of salt used in the extraction medium is typically selected so as not to interfere with any of the reactions in which the extract may be used.

Typically, the salt is either a monovalent salt, a divalent salt, or some combination thereof. Preferably, the salt is a monovalent salt. Exemplary monovalent salts include lithium fluoride (LiF), lithium chloride (LiCl), lithium bromide (LiBr), lithium iodide (LiI), sodium fluoride (NaF), sodium chloride (NaCl), sodium bromide (NaBr), sodium iodide (NaI), potassium fluoride (KF), potassium chloride (KCl), potassium bromide (KBr), potassium iodide (KI), rubidium fluoride (RbF), rubidium chloride (RbCl), rubidium bromide (RbBr), rubidium iodide (RbI), cesium fluoride (CsF), cesium chloride (CsCl), cesium bromide (CsBr), and cesium iodide (CsI), among others. Preferably, the monvalent salt is selected from the group consisting of NaF, NaCl, NaBr, NaI, KF, KCl, KBr, KI, and combinations thereof. More preferably, the monovalent salt is NaCl or KCl.

In certain embodiments, the extraction solution may comprise a divalent salt. Exemplary divalent salts include beryllium chloride (BeCl₂), beryllium fluoride (BeF₂), beryllium bromide (BeBr₂), beryllium iodide (BeI₂), magnesium chloride (MgCl₂), magnesium fluoride (MgF₂), magnesium bromide (MgBr₂), magnesium iodide (MgI₂), calcium chloride (CaCl₂), calcium fluoride (CaF₂), calcium bromide (CaBr₂), calcium iodide (CaI₂), strontium chloride (SrCl₂), strontium fluoride (SrF₂), strontium bromide (SrBr₂), strontium iodide (SrI₂), barium chloride (BaCl₂), barium fluoride (BaF₂), barium bromide (BaBr₂), and barium iodide (BaI₂), among others. Preferably the divalent salt is selected from the group consisting of MgCl₂, MgF₂, MgBr₂, MgI₂. More preferably, the divalent salt is MgCl₂.

It will be recognized by those skilled in the art that some monovalent and/or divalent salts may have an inhibitory effect on PCR. Therefore, in certain instances, it may be advantageous to add a chelating agent, as discussed below, to the cellular extract after RNA has been extracted and prior to performing PCR to control the amount of inhibitory monovalent and/or divalent salt in a PCR or RT-PCR reaction mixture.

As discussed above, the salt in the extraction medium acts to render the medium hypertonic, thus enabling RNA to be released from the ruptured cells by way of osmotic pressure exerted on the cell membrane or cell wall. It is thus preferable that the amount of salt present in the extraction medium be sufficient to render the medium hypertonic (i.e., the concentration of salt in the extraction medium is higher than that in the cell). The concentration of salt in the extraction medium is preferably from about 150 mM to about 5M, more preferably from about 200 mM to about 3.5 M, and still more preferably is about 300 mM. If the extraction medium comprises a divalent salt, generally a lower amount of salt will be needed than if the extraction medium comprises a monovalent salt. For example, if the extraction medium comprises a divalent salt, the concentration of the salt in the extraction medium is preferably from about 10 mM to about 5 M, and more preferably is about 25 mM to about 100 mM.

Optionally, the extraction medium may also comprise certain agents to aid in stabilizing the extracted RNA. Typically, these agents and their concentrations are chosen so as not to interfere with reverse transcription, PCR, and/or other reactions in which the extract may be used. For example, in one embodiment, the extraction medium may comprise at least one RNase inhibitor. RNase inhibitors are known to those of skill in the art and include proteinacious RNase inhibitors such as human placental RNase inhibitors and porcine liver RNase inhibitors, anti-nuclease antibodies such as ANTI-RNase (Ambion, Inc., Austin, Tex.), clays such as macaloid and bentonite, polyanions, nucleotide analogs, reducing agents such as β-mercaptoethanol, dithiothreitol (DTT), dithioerythritol (DTE), and glutathione, and other such inhibitors. Preferably, the RNase inhibitor is a proteinacious RNase inhibitor. The RNase inhibitor may be present in the extraction medium in an amount of from about 0.00001 units/μl to about 1,000 units/μl, and more preferably about 0.0001 units/μl to about 1 unit/μl. RNase inhibitors are particularly useful when the cell from which the RNA is to be extracted is located in a tissue, which may have more RNase activity than an individual cell or cell suspension. The RNase inhibitors may optionally be contained in the extraction medium and/or may be added to the cellular extract after extraction.

Other agents that may be optionally included in the extraction medium or added to the extract after extraction include various molecules that selectively degrade DNA such as DNase I, Bal31 nuclease, T7 endonuclease, Neurospora crassa nuclease, Lambda exonuclease, Exonuclease I, Exonuclease III, and Exonuclease VII, as well as agents that release cells from tissues, such as cellulases, pectinases, amylases, oxalyticase, zymolyase, lysozyme, and the like. Such agents may be present in the extraction medium in an amount of from about 0.00001 units/μl to about 1,000 units/μl, and more preferably about 0.0001 units/μl to about 1 unit/μl.

In addition to the salt and detergent, the extraction medium may optionally comprise one or more buffering agents, the selection and use of which can be readily determined by one skilled in the art. Preferably, the extraction medium has a pH of from about 3 to about 10, and more preferably has a pH of about 7 to 8. As such, it is generally preferably that any buffer present in the extraction medium be suitable for maintaining the pH within this range. Typically, the buffer is prepared from a substance having a pKa value from one unit less than to one unit greater than the desired pH. Thus, for example, a pH 8.0 buffer can be prepared using a substance having a pKa from about 7.0 to 9.0. Examples of suitable buffers components include N-(2-Acetamido)-2-aminoethanesulfonic acid (ACES) (pKa about 6.8 at 25° C.), acetate (pKa about 4.7 at 20° C.), acetic acid, N-(2-Acetamido)iminodiacetic acid (ADA) (pKa about 6.6 at 25° C.), 2-Amino-2-methyl-1-propanol (AMP) (pKa about 9.7 at 25° C.), 2-Amino-2-methyl-1,3-propanediol (AMPD) (pKa about 8.8 at 25° C.), N-(1,1-Dimethyl-2-hydroxyethyl )-3-amino-2-hydroxypropanesulfonic acid (AMPSO) (pKa about 9.0 at 25° C.), N,N-Bis(2-hydroxyethyl)-2-aminoethanesulfonic acid (BES) (pKa about 7.1 at 25° C.), bicarbonate (pKa about 6.35 at 20° C.), N,N-Bis(2-hydroxyethyl) glycine (Bicine) (pKa about 8.3 at 25° C.), BIS-TRIS (pKa about 6.5 at 25° C.), BIS-TRIS propane (pKa1 about 6.8 and pKa2 about 9.0 at 25° C.), 4-(Cyclohexylamino)-1-butanesulfonic acid (CABS) (pKa about 10.7 at 25° C.), 3-(Cyclohexylamino)propanesulfonic acid (CAPS) (pKa about 10.4 at 25° C.), 3-(Cyclohexylamino)-2-hydroxy-1-propanesulfonic acid (CAPSO) (pKa about 9.6 at 25° C.), carbonate (pKa about 10.3 at 20° C.), cyclohexanediamine tetraacetic acid (CDTA), 2-(N-Cyclohexylamino)ethanesulfonic acid (CHES) (pKa about 9.3 at 25° C.), monovalent citrate (pKa about 3.09 at 20° C.), divalent citrate (pKa about 4.75 at 20° C.), trivalent citrate (pKa about 5.41 at 20° C.), citric acid (pKa1 about 3.13; pKa2 about 4.76; pKa3 about 6.4), 3-(N,N-Bis[2-hydroxyethyl]amino)-2-hydroxypropanesulfonic acid (DIPSO) (pKa about 7.6 at 25° C.), ethylenediaminetetraacetic acid (EDTA), (ethylenebis(oxyethylenenitrilo))tetraacetic acid (EGTA), glycinamide*HCl (pKa about 8.2 at 20° C.), glycine (pKa about 2.35), gly-gly (pKa about 8.2 at 25° C.), N-(2-Hydroxyethyl)piperazine-N′-(4-butanesulfonic acid) (HEPBS) (pKa about 8.3 at 25° C.), N-(2-Hydroxyethyl)piperazine-N′-2-ethanesulfonic acid (HEPES) (pKa about 7.5 at 25° C.), N-(2-Hydroxyethyl)piperazine-N′-(3-propanesulfonic acid) (HEPPS) (pKa about 8.0 at 25° C.), β-Hydroxy-4-(2-hydroxyethyl)-1-piperazinepropanesulfonic acid monohydrate (HEPPSO) (pKa about 7.8 at 25° C.), maleic acid (pKa about 2.0), malic acid (pka1 about 3.3; pKa2 about 5.1), 2-(N-Morpholino)ethanesulfonic acid (MES) (pKa about 6.1 at 25° C.), 4-(N-Morpholino)butanesulfonic acid (MOBS) (pKa about 7.6 at 25° C.), morpholinopropane sulfonic acid (MOPS) (pKa about 7.2 at 25° C.), β-Hydroxy-4-morpholinepropanesulfonic acid (MOPSO) (pKa about 6.9 at 25° C.), N-lauryl sarcosine, 1,4-Piperazinediethanesulfonic acid (PIPES) (pKa about 6.8 at 25° C.), Piperazine-1,4-bis(2-hydroxypropanesulfonic acid) dehydrate (POPSO) (pKa about 7.8 at 25° C.), potassium acetate (pKa about 4.8), sodium acetate (pKa about 4.7), N-tris(Hydroxymethyl)methyl-4-aminobutanesulfonic acid (TABS) (pKa about 8.9 at 25° C.), N-[Tris(hydroxymethyl)methyl]-3-aminopropanesulfonic acid (TAPS) (pKa about 8.4 at 25° C.), N-[Tris(hydroxymethyl)methyl]-3-amino-2-hydroxypropanesulfonic acid (TAPSO) (pKa about 7.6 at 25° C.), tetraethylammonium (TEA) (pKa about 7.8 at 25° C.), N-Tris(hydroxymethyl)methyl-2-aminoethanesulfonic acid (TES) (pKa about 7.4 at 25° C.), N-[Tris(hydroxymethyl)methyl]glycine (Tricine) (pKa about 8.1 at 25° C.), Tris, and Trizma® Buffer solution (Sigma-Aldrich, Co., St. Louis, Mo.) (pKa about 8.1 at 25° C.), among others. Preferably, the buffer is Tris at pH 8.0. Buffers from other substances can be readily prepared by those skilled in the art. As will be recognized by those skilled in the art, some of these buffers (such as EDTA and EGTA), may also act as chelating agents, as discussed below. In instances where these buffers are used, it may be necessary to adjust the concentration of salt in the extraction medium to take into account the chelating action of these buffers.

The buffer may optionally be contained in the extraction medium and/or may be added to the extract after extraction. For example, in one embodiment, the extraction medium may comprise from about 0.1 mM to about 5 M buffer, more preferably about 0.1 mM to about 500 mM buffer, and more preferably about 100 mM buffer.

The extraction medium may also include other components that aid in extraction, such as glycerol, a preservative such as Kathon™ preservative, and/or bovine serum albumin (BSA), among others.

In one preferred embodiment, the extraction medium comprises 1% by weight of a non-ionic detergent, 300 mM of a monovalent salt, 100 mM of a buffer, and 5% by weight glycerol. Preferably, the non-ionic detergent is Triton X-100, the monovalent salt is NaCl, and the buffer is Tris, pH 8.0.

As noted above, the extraction may advantageously be performed without the use of certain catabolic enzymes commonly used to lyse cells. Thus, in some preferred embodiments, these enzymes are not present in the extraction medium or are only present in limited amounts. Examples of such catabolic enzymes include proteases, such as proteinase K or lyticase, enzymes that degrade lipids and/or fats, such as lipase, enzymes that degrade carbohydrates, such as amylase or cellulase, and other catabolic enzymes, such as lysozyme, pectinase, and any other enzymes or molecules that degrade connective tissue and that do not inhibit enzymatic reactions, specifically reverse transcription or PCR. In one particular embodiment, the catabolic enzyme is a protease, such as a serine protease like trypsin, chymotrypsin, elastase, subtilisin, streptogrisin, thermitase, aqualysin, and carboxypeptidase A, D, C, or Y; a cystein protease like papain and chlostripain; an acid protease like pepsin, chymosin, and cathepsin; a metalloprotease like pronase, thermolysin, collagenase, dipase; an aminopeptidase; and/or carboxypeptidase A, B, E/H, M, T, or U.

Preferably, these catabolic enzymes are either not present in the extraction medium, or present at low concentrations. Thus, the catabolic enzymes, if present in the extraction medium, are preferably at a concentration of about 0.0001 units/mL or less, more preferably about 0.0000001 units/mL or less for each enzyme, and still more preferably the extraction medium is free of these catabolic enzymes.

In certain embodiments, the extraction medium may optionally further comprise reagents needed for reverse transcription. Such reagents include any of those described herein as suitable for use in the RT composition. In one preferred embodiment, the extraction medium further comprises MgCl₂, dNTPs, a reverse transcriptase such as murine leukemia virus reverse transcriptase (MMLV-RT), an RNase inhibitor such as those described herein, and a primer such as oligo dT, random hexamers, nonamers, or decamers, or gene specific primers (GSP). Any suitable amount of these reagents may be used. In one preferred embodiment, this supplemented extraction medium further comprises about 3 mM MgCl₂, about 0.2 mM each dNTP, about 1 unit/μl of MMLV-RT, about 0.4 units/μl of an RNase inhibitor, about 5 mM DTT, and about 1 μM to about 5 μM primer. In this embodiment, the supplemented extraction medium is contacted with a cell population to extract RNA, and reverse transcription may be performed directly on the extract without adding any further reagents. In this embodiment, the supplemented extraction medium is typically contacted with the sample for about 0.01 minutes to about 1500 minutes, and preferably for about 15 minutes to extract the RNA and perform reverse transcription. The incubation temperature typically ranges from ambient temperature (i.e., room temperature) to about 45° C. qPCR may then be performed on the RT reaction product under suitable conditions, as described herein.

Optionally, in certain embodiments, the extraction medium may further comprise reagents needed for one-step RT-PCR. Such reagents include any of those described herein as suitable for use in the RT-PCR composition including, for example, primers, dNTPs, appropriate buffers, DNA polymerase, detection dyes or probes, a divalent salt such as MgCl₂, and/or a reverse transcriptase, among others. In this embodiment, the supplemented extraction medium may be contacted with a cell population to extract RNA, and one-step RT-PCR may be performed directly on the extract without adding any further reagents.

The extraction medium is typically an aqueous solution, however, in certain embodiments, the extraction medium can be in the form of an aqueous dispersion, suspension, emulsion, or the like, or may be in a dry form such as a powder. Preferably, the extraction medium has a pH of from about 3 to about 10, and more preferably from about 7 to about 9.

The extraction medium may be one composition or, optionally, can be two or more compositions that are mixed together to form the extraction medium. For example, all components of the extraction medium may be in one composition, and that composition may be contacted with the cell population to form a cellular extract. Alternately, the components of the extraction medium may be in two or more compositions, and these compositions may be combined with the cell population to form a cellular extract. For example, in one embodiment, the extraction medium may contain a salt component and a detergent component, and each component may be separately added to the cell population to form the cellular extract. Likewise, any optional reagents (e.g., RNase inhibitors, buffers, etc.) used for extraction may be present as part of one extraction medium, or alternately may be added to the cell population along with the other components to form the cellular extract.

As will be apparent to those skilled in the art, the amount of extraction medium needed to perform the extraction will vary depending on the amount and type of cells in the cell population. The amount of extraction medium should, however, be sufficient to effectively release RNA from the desired number of cells. Typically, a ratio of 1:500 (μl extraction medium:number of cells) is sufficient. For example, about 100 μl of extraction medium is typically sufficient to perform an extraction on a cell population comprising about 50,000 cells, and about 200 μl of extraction medium is typically sufficient to perform an extraction on a cell population comprising about 100,000 cells.

In general, it is not necessary to contact the cell population with the extraction medium for an extended period of time to achieve RNA extraction since RNA is typically released from the cells upon contact with the extraction medium. For example, the extraction medium is typically contacted with the cell population for at least about 0.01 minutes, and preferably for about 10 minutes. Optionally, the cell population may be incubated in the extraction medium. Incubation is preferably for about 60 minutes or less, more preferably about 45 minutes or less, still more preferably about 30 minutes or less, and still more preferably about 10 minutes or less.

The temperature for incubation will depend upon the cell population being extracted. For example, for mammalian tissues, extraction is preferably at room temperature or higher, and preferably is at least about 20° C. For other source materials, where enzymes are used to release RNA, extraction temperatures may vary.

During incubation, the cell population and the extraction medium may optionally be agitated. Agitation helps increase the contact between the cells in the cell population and the extraction medium, and allows for more uniform distribution of the extraction medium throughout the cell population. Agitation may be done by any method known in the art, including shaking, stirring, mixing, vortexing, and pipetting up and down, among others.

Advantageously, the extraction media described herein may be used to extract RNA from cells without the use of enzymes, freezing or heating steps, centrifugation, or other such traditional extraction means.

### Two-Step RT-PCR

The cellular extract produced from the extraction procedure, described above, may be used directly in a number of different reactions. In one embodiment, the RNA in the cellular extract may be subjected to reverse transcription alone or to reverse transcription and polymerase chain reaction. When used together, reverse transcription and polymerase chain reaction may be performed sequentially in two steps, or together in one step with all reaction composition reagents being added to the cellular extract.

For example, in one embodiment, a two-step RT-PCR reaction may be performed using the cellular extract. In this embodiment, all or a portion of the cellular extract may be combined with a reverse transcription (RT) composition to form an RT reaction mixture. As mentioned above, an RT composition typically comprises some or all of the reagents needed to synthesize a DNA product from an RNA template, in this instance, the extracted RNA present in the cellular extract. Suitable reagents for performing reverse transcription are known to those skilled in the art and include, for example, a primer that hybridizes to the RNA template to prime the synthesis of the copy of DNA, dNTPs, a divalent salt such as MgCl₂, and appropriate buffers such as Tris-HCl, pH 8.3. Optionally, the RT composition may also comprise, an RNase inhibitor such as those described herein, dithiothreitol (DTT), glycerol, a preseravitive such as Kathon™ preservative, surfactants or facilitators such as the surfactant glycolic acid ethoxylate oleyl ether (GAEOE) or BSA, and/or a reverse transcriptase. Other reagents useful in performing reverse transcription will be readily apparent to those skilled in the art, and may be used without departing from the scope of this invention.

Suitable primers may be designed by one skilled in the art to prime the synthesis of a copy of DNA using the RNA as template in the reverse transcription reaction. Examples of primers that may be included in the RT composition include but are not limited to random primers such as hexamers, nonamers, or decamers, gene specific primers, oligo dT, and mixtures thereof. More than one primer may be included if it is desired to make DNA copies from more than one target RNA.

Any number of a variety of reverse transcriptases may be used including Thermus thermophilus reverse transcriptase (Tth-RT), Rous Sarcoma-reverse transcriptase, avian myeloblastosis virus reverse transcriptase (AMV-RT), Moloney murine leukemia virus reverse transcriptase (MMLV-RT), or any modified reverse transcriptase such as Superscript™ RNase H-reverse transcriptase (Invitrogen Corp., Carlsbad, Calif.). Preferably, the reverse transcriptase is MMLV-RT. The reverse transcriptase may be included as a part of the RT composition or, alternately, may be added to the cellular extract separately from the RT composition, as discussed herein.

In one preferred embodiment, the RT composition of the present invention comprises dNTPs, random nonamers, an RNase inhibitor, MgCl₂, DTT, and Tris-HCl, pH 8.3. Optionally, an MMLV-RT may be included in the RT composition or, alternately, may be added to the cellular extract separately from the RT composition.

The amount of each component needed to perform reverse transcription is known to or is readily ascertainable by those skilled in the art. For example, standard amounts of reagents used in reverse transcription reactions include: about 50 mM Tris-HCl, pH 8.3, about 1 mM to about 5 mM DTT, about 0.5 units/μl to about 10 unit/μl of an RNase inhibitor, about 400 μM to about 600 μM of each dNTP, about 5 mM to about 15 mM of MgCl₂, about 0.5 units/μl to about 1 unit/μl of a reverse transcriptase, and a primer selected from the group consisting of about 1 μM to about 5 μM nanomers, about 0.5 μM to about 1 μM gene specific primers, about 1 μM to about 5 μM oligo dT, and combinations thereof. It will be apparent to those skilled in the art that the reagents and amount of each reagent in the RT composition may vary considerably from those described herein and still result in a suitable composition for performing reverse transcription. Furthermore, the concentrations of these reagents may optionally be scaled up or down, depending on the amount of template RNA to be used in the reaction.

As will be apparent to those skilled in the art, compositions traditionally used to perform reverse transcription or PCR typically comprise monovalent and divalent salt. However, a concentration of available monovalent or divalent salt that is too high may interfere with the reverse transcription or PCR reaction. As discussed above, the extraction medium used to produce the cellular extract of the present invention preferably comprises from about 150 mM to about 5 M of a monovalent salt (or 10 mM to about 5 M of a divalent salt). When the cellular extract is used in a reverse transcription reaction, some of the monovalent and/or divalent salt present in the extraction medium is present in the RT reaction mixture. Consequently, it may be advantageous to limit the amount of available monovalent and/or divalent salt in the RT composition to avoid a high cumulative available monovalent or divalent salt concentration in the RT reaction mixture. The same may be said for the PCR compositions and reaction mixtures, described herein.

Thus, the type and concentration of salt in the RT composition will typically depend on the type and amount of salt in the extraction medium and the amount of extract and RT composition used. For example, in instances where the extraction medium comprises the preferred level of a monovalent salt, the cellular extract produced using the extraction medium will typically comprise a sufficient amount of available monovalent salt to perform reverse transcription, and additional amounts of monovalent salt are generally not needed. The RT composition may, however, optionally comprise monovalent and/or divalent salt. In instances where the extraction medium comprises a divalent salt, the cellular extract produced using the extraction medium will typically comprise a sufficient amount of available divalent salt to perform reverse transcription, and additional amounts of divalent salt are generally not needed.

It is thus preferable that the total amount of available monovalent or divalent salt in the RT reaction mixture (i.e., the mixture of the cellular extract and the RT composition) be an amount sufficiently low that it does not interfere with reverse transcription and/or PCR. Preferably, the total amount of available monovalent salt in the RT reaction mixture is about 75 mM or less, and more preferably is about 50 mM. Preferably, the total amount of available divalent salt in the RT reaction mixture is about 10 mM or less. It will be appreciated that the actual amount of available monovalent and/or divalent salt in the RT composition may vary considerably depending on the amount of available salt provided by the cellular extract and the amount of cellular extract used to form the RT reaction mixture. In one embodiment, the RT composition is free of available monovalent salt. In another embodiment, the RT composition is free of available divalent salt.

In certain embodiments, the amount of available monovalent and/or divalent salt in the RT reaction mixture may be controlled using a chelating agent. For example, certain compounds, such as crown ethers, are known to chelate alkali metal cations, such as sodium and potassium. In general, crown ethers are heterocyclic chemical compounds that are cyclic oligomers of ethylene oxide. The essential repeating unit of any simple crown ether is ethyleneoxy (i.e., —CH₂CH₂O—), which repeats twice in dioxane, four times in 12-crown-4, five times in 15-crown-5, six times in 18-crown-6, and so forth. Macrocylces of (—CH₂CH₂O—)ₙ in which n≧4 are generally referred to as crown ethers because the molecules formed when this group of heterocycles binds to cations resemble a crown sitting on a head in structure.

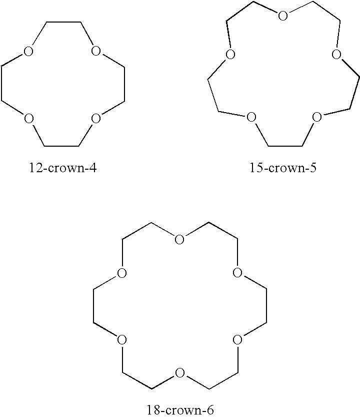

The crown ethers are known to strongly solvate cations. The oxygen atoms of the crown ether coordinate with a cation in the interior of the ring, while the exterior of the ring is hydrophobic. As a result, a cation complexed with the crown ether is soluble in nonpolar solvents. The size of the cation that may be solvated is determined by the size of the interior of the crown ether. For example, 18-crown-6 has a high affinity for potassium cation, 15-crown-5 has an affinity for sodium cation, and 12-crown-4 has an affinity for lithium cation. The amount of available monovalent and/or divalent salt in the RT reaction mixture may be controlled by adding a chelating agent, such as a crown ether, to the cellular extract. Optionally, other chelating agents may also be used, such as EDTA, EGTA, nitrilotriacetic acid (NTA), porphine, diethylenetriaminepentaacetic acid (DTPA), and the like. When the salt in the RT reaction mixture is a divalent salt, the chelating agent may preferably be EDTA, EGTA, NTA, porphine, and/or DTPA. Adding a chelating agent to the cellular extract may help prevent the monovalent or divalent salt from interfering in the reverse transcription reaction. Optionally, the chelating agent may be present as part of the RT composition and/or a PCR composition, described below.

The amount of chelating agent used will typically depend on the concentration of monovalent or divalent salt in the reaction mixture. In general, the amount of chelating agent present in the reaction mixture will be about the same as the amount of salt in excess of the desired salt concentration in the reaction mixture. For example, as mentioned above, it is typically preferable that the total amount of available monovalent salt in the RT reaction mixture be about 75 mM or less, and more preferably be about 50 mM, and the total amount of available divalent salt in the RT reaction mixture be about 10 mM or less. Thus, in instances where the preferred amount of available monovalent salt is about 75 mM or less, the amount of chelating agent having an affinity for monovalent cations in the RT reaction mixture will preferably equal the amount of available monovalent salt in excess of 75 mM in the RT reaction mixture. Likewise, in instances where the preferred amount of available divalent salt is about 10 mM or less, the amount of chelating agent having an affinity for divalent cations in the RT reaction mixture will preferably equal the amount of available divalent salt in excess of 10 mM in the RT reaction mixture.

Like discussed above for the extraction medium, the RT composition may be one composition or, optionally, can be two or more compositions that are mixed together to form the RT composition. For example, all reagents needed to perform reverse transcription may be in one composition, and that composition may be contacted with the cellular extract to form an RT reaction mixture. Alternately, the reagents needed to perform reverse transcription may be in two or more separate compositions, and these compositions may be combined and/or contacted with the cellular extract to form the RT reaction mixture. For example, in one embodiment, the RT composition may not comprise a reverse transcriptase. In this instance, the reverse transcriptase and the RT composition may be separately added to the cellular extract to form the RT reaction mixture.

As will be apparent to those skilled in the art, the amount of cellular extract needed to perform the reverse transcription will vary depending on the amount of target RNA in the extract. Typically, the amount of RNA template needed to perform reverse transcription is at least about 50 copies or molecules.

The reverse transcription reaction typically consists of a single temperature incubation at a temperature of between about 37° C. and about 95° C. Different temperatures are appropriate for different reverse transcriptase enzymes and different primers, as is known to one skilled in the art. Generally, the RT reaction mixture is incubated for about 10 minutes to about 2 hours to form an RT product comprising cDNA.

In the second part of the two-step RT-PCR reaction, all or a portion of the product of the reverse transcription reaction may be contacted with a PCR composition to form a PCR reaction mixture. As mentioned herein, a PCR composition typically comprises some or all of the reagents needed to amplify a DNA template, in this instance, the cDNA present in the RT reaction product. Suitable reagents for performing PCR are known to those skilled in the art and include, for example, a pair of nucleic acid primers that initiate synthesis of the desired segment of DNA from the reverse transcribed template, dNTPs, a DNA polymerase such as Taq polymerase, Deep Vent® DNA polymerase (New England Biolabs, Inc., Ipswich, Mass.), Klen Taq® LV DNA polymerase, and Pyrococcus furiosus (Pfu) DNA polymerase, and buffers such as Tris-HCl, pH 8.3 and/or other suitable buffers, such as those listed above. Optionally, the PCR composition may further comprise detection dyes or probes such as TaqMan® probes (Applied Biosystems, Inc., Foster City, Calif.), Eclipse® probe (Nanogen), Pleiades probe (Nanogen), Universal ProbeLibrary (Roche Applied Science), Molecular Beacon, Scorpion probes, fluorogenic probes, and/or dyes that specifically bind to double stranded DNA (dsDNA) such as intercalating dyes like SYBR® Green I, and minor groove binding dyes; an RNase inhibitor such as those described herein; a divalent salt such as MgCl₂; a monovalent salt such as KCl; a Taq antibody such as JumpStart™ Taq antibody (available from Sigma-Aldrich, Co., St. Louis, Mo.); a chelating agent such as crown ethers, EDTA, and the like; glycerol; a preseravitive such as Kathon™ preservative; GAEOE; and/or various stabilizers or facilitators such as BSA, Tween 20, and Triton X-100. Other reagents useful in performing PCR will be readily apparent to those skilled in the art, and may be used without departing from the scope of this invention.

In one preferred embodiment, the PCR composition of the present invention comprises MgCl₂, dNTPs, JumpStart™ Taq polymerase, an RNase inhibitor, KCl, and Tris-HCl, pH 8.3. Optionally, a JumpStart™ Taq antibody and/or a SYBR® Green I dye may also be included in the PCR composition.

The amount of each component needed to perform PCR is known to or is readily ascertainable by those skilled in the art. For example, standard amounts of reagents used in PCR include: about 10 mM of Tris-HCl, pH 8.3, about 50 mM of a monovalent salt such as KCl, about 10 μM to about 400 μM of each dNTP, about 0.1 μM to about 1 μM of each primer, about 0.5 units/50 μl to about 2.5 units/50 μl of polymerase, about 1 mM to about 4 mM of MgCl₂, and about 0.5 to about 1.5 units/ml of RNase inhibitor. It will be apparent to those skilled in the art that the reagents and amount of each reagent in the PCR composition may vary considerably from those described herein and still result in a suitable composition for performing PCR. Furthermore, the concentrations of these reagents may optionally be scaled up or down, depending on the amount of template DNA to be used in the reaction.

As discussed above, a concentration of available monovalent or divalent salt that is too high may interfere with a PCR reaction. It is thus preferable that the total amount of available monovalent or divalent salt in the PCR reaction mixture be an amount sufficiently low that it does not interfere with a PCR reaction. Preferably, the total amount of available monovalent salt in the PCR reaction mixture is about 75 mM or less, and more preferably is about 50 mM. Preferably, the total amount of available divalent salt in the PCR reaction mixture is about 10 mM or less. The salt may come from the RT product or may be present in the PCR composition. It will be appreciated that the actual amount of available monovalent and/or divalent salt in the PCR composition, if any, may vary considerably depending on the amount of available salt provided by the RT reaction product and the amount of RT reaction product used to form the PCR reaction mixture. In one embodiment, the PCR composition is free of available monovalent salt. In another embodiment, the PCR composition is free of available divalent salt.

A chelating agent such as those described above, may also be added to the PCR reaction mixture to control the amount of available monovalent and/or divalent salt in the PCR reaction mixture. The amount of chelating agent in the PCR composition will typically depend on the concentration of monovalent or divalent salt in the composition, and generally will be about the same as the amount of salt in excess of the desired salt concentration in the composition, as discussed above.

Like discussed above for the extraction medium and the RT composition, the PCR composition may be one composition or, optionally, can be two or more compositions that are mixed together to form the PCR composition. For example, all reagents needed to perform PCR may be in one composition and that composition may be contacted with the RT reaction product to form a PCR reaction mixture. Alternately, the reagents needed to perform PCR may be in two or more separate compositions, and these compositions may be combined and/or contacted with the RT reaction product to form the PCR reaction mixture.

As will be apparent to those skilled in the art, the amount of RT reaction product needed to perform PCR will vary depending on the amount of cDNA in the RT reaction product. Typically, the amount of template DNA needed to perform PCR is at least about 10 copies or molecules.

PCR uses thermocycling to amplify a DNA template. PCR typically consists of repeated cycles of template denaturation (e.g., denaturation of the cDNA), primer annealing, and extension of the annealed primers. The DNA template is typically denatured at a temperature greater than about 90° C., and more typically at a temperature of about 94° C. to about 96° C. After the DNA strands have separated, the temperature is lowered so that the primers can attach to the single DNA strands (i.e., primer annealing). The primer annealing temperature is dependent on the melting temperature of the specific primers used in the reaction, and is usually about 5° C. below the primer melting temperature, typically about 45° C. to about 65° C. The temperature required for extension depends on the DNA-polymerase used, and the time required for this step depends on the DNA-polymerase and on the length of the DNA fragment to be amplified.

### One-Step RT-PCR

In another embodiment, a one-step RT-PCR reaction may be performed using the cellular extract. In this embodiment, all or a portion of the cellular extract may be combined with an RT-PCR composition to form an RT-PCR reaction mixture. The RT-PCR reaction mixture is incubated to allow reverse transcription to occur, as described above. The resulting product is ready for use in a PCR reaction. Because all the reagents required for both reverse transcription and PCR may be included in the RT-PCR reaction mixture, there is no need to add further reagents prior to PCR.

The RT-PCR composition may comprise any of the reagents listed above as suitable for use in the RT composition or the PCR composition. Typically, the RT-PCR composition comprises primers, dNTPs, appropriate buffers, MgCl₂, and a DNA polymerase. Optionally, the RT-PCR composition may also comprise detection dyes or probes, an RNase inhibitor, a chelating agent such as those described herein, and/or a reverse transcriptase.

In addition to the components listed above, the RT-PCR composition of the present invention may optionally comprise other reagents that may facilitate RT-PCR such as glycerol, bovine serum albumin (BSA), the surfactant glycolic acid ethoxylate oleyl ether (GAEOE), and a preservative such as Kathon™ preservative.

In one preferred embodiment, the RT-PCR composition of the present invention comprises MgCl₂, dNTPs, glycerol, a polymerase such as JumpStart™ Taq polymerase, BSA, an RNase inhibitor, a preservative such as Kathon™ preservative, GAEOE, and a buffer such as Tris-HCl, pH 8.3. Optionally, a MMLV-RT may be included in the RT-PCR composition or, alternately, may be added to the cellular extract separately from the RT-PCR composition.

The amount of each component needed to perform one-step RT-PCR is known to or is readily ascertainable by those skilled in the art. For example, standard amounts of reagents used in one-step RT-PCR reactions include: about 10 mM Tris-HCl, pH 8.3, about 0.3 units/μl to about 0.5 units/μl of an RNase inhibitor, about 200 μM to about 400 μM of each dNTP, about 0.1 μM to about 1 μM of each primer, about 0.04 units/50 μl to about 0.4 units/50 μl of a reverse transcriptase, and about 1.5 mM to about 10 mM of MgCl₂.

In certain embodiments, the RT-PCR composition may also comprise about 5% to about 10% by weight glycerol, about 0.025% to about 0.1% by weight BSA, about 0.025 units/μto about 0.75 units/pl of a polymerase, about 0.025 ppm to about 0.05 ppm of a preservative like Kathon™ preservative, and about 0.2% to about 0.4% by weight of GAEOE.

As discussed above for the RT composition, the type and concentration of salt in the RT-PCR composition will typically depend on the type and amount of salt in the extraction medium. It is generally preferable that the total amount of available monovalent or divalent salt in the RT-PCR reaction mixture (i.e., the mixture of the cellular extract and the RT-PCR composition) be an amount sufficiently low that it does not interfere with reverse transcription and/or a PCR reaction. Preferably, the total amount of available monovalent salt in the RT-PCR reaction mixture is about 75 mM or less, and more preferably is about 50 mM. Preferably, the total amount of available divalent salt in the RT-PCR reaction mixture is about 10 mM or less. It will be appreciated that the actual amount of available monovalent and/or divalent salt in the RT-PCR composition may vary considerably depending on the amount of available salt provided by the cellular extract and the amount of cellular extract used to form the RT-PCR reaction mixture. In one embodiment, the RT-PCR composition is free of available monovalent salt. In another embodiment, the RT-PCR composition is free of available divalent salt.

Optionally, as discussed above for two-step RT-PCR, the amount of available monovalent and/or divalent salt in the RT-PCR reaction mixture may be controlled using a chelating agent such as those described herein. The chelating agent may be added to the RT-PCR reaction mixture, or optionally, may be present as part of the RT-PCR composition. As discussed above, the amount of chelating agent used will typically depend on the concentration of monovalent or divalent salt in the reaction mixture, and generally will be about the same as the amount of salt in excess of the desired salt concentration in the reaction mixture.

Like discussed above for the extraction medium, the RT composition, and the PCR composition, the RT-PCR composition may be one composition or, optionally, can be two or more compositions that are mixed together to form the RT-PCR composition. For example, all reagents needed to perform one-step RT-PCR may be in one composition, and that composition may be contacted with the cellular extract to form an RT-PCR reaction mixture. Alternately, the reagents needed to perform one-step RT-PCR may be in two or more separate compositions, and these compositions may be combined and/or contacted with the cellular extract to form the RT-PCR reaction mixture. For example, in one embodiment, the RT-PCR composition may not comprise a reverse transcriptase. In this instance, the reverse transcriptase and the RT-PCR composition may be separately added to the cellular extract to form the RT-PCR reaction mixture.

As will be apparent to those skilled in the art, the amount of cellular extract needed to perform one-step RT-PCR will vary depending on the amount of RNA in the extract. Typically, the amount of RNA template needed to perform one-step RT-PCR is at least about 50 copies or molecules.

In addition to the methods described herein, the cellular extract containing RNA may be used in a variety of different reactions including, for example, direct labeling and hybridization (e.g. microarray, Northern blot, etc.) reactions, reverse transcription labeling and hybridization reactions, immunoprecipitation or hybrid selection of RNA-protein pairs, and the like.

### Kits

Another aspect of the present invention is a kit comprising reagents for forming an extraction medium. The kits may be used to extract RNA from cells and optionally prepare cDNA according to the methods of the present invention.

The kits of the present invention may comprise reagents for forming an extraction medium, and instructions for using the reagents and kit. The reagents may comprise a detergent and salt, such as those described herein as suitable for formation of an extraction medium. Optionally, the kit may further provide additional reagents suitable for use in the extraction medium, such as RNase inhibitors, DNA degrading agents, and buffering agents, among others. The reagents may be provided as a single composition or, optionally, can be provided separately as two or more compositions that may be combined to form an extraction medium. The reagents are provided in the kit in amounts sufficient to form the desired extraction medium.

The kits may also comprise a reverse transcriptase and reagents for performing reverse transcription and/or RT-PCR. For instance, the kit may comprise reagents, such as those described herein, that may be used to form an RT composition, a PCR composition, and/or an RT-PCR composition. The RT, PCR, and/or RT-PCR compositions may be provided as one composition or, optionally, can be provided as two or more compositions that may be combined to form the RT, PCR, and/or RT-PCR composition. Suitable reagents for use in such compositions and that may be included in the kits of the present invention are described above. Such reagents are provided in the kit in amounts sufficient to form the desired composition.

The kit may further comprise instructions for using the reagents and kit. For instance, the instructions may describe how to form an extraction medium, how to form a RT composition, how to form a PCR composition, and/or how to form a RT-PCR composition of the present invention. In one particular embodiment, the instructions describe how to form an extraction medium comprising about 0.1% to about 10% by weight of a detergent and about 10 mM to about 5 M of a salt. The instructions may describe how to form extraction mediums having any suitable volume. In one non-limiting example, the instructions may describe how to form an extraction medium having a volume ranging from a microliter to several gallons. The instructions may also optionally describe how to extract RNA from cells and how to perform reverse transcription, PCR, and/or RT-PCR. Typically, the instructions included in the kit will be written instructions.

### Definitions

As used herein, the term “RNA” refers to a nucleic acid molecule comprising a ribose sugar as opposed to a deoxyribose sugar as found in DNA. As used herein, RNA refers to all species of RNA including messenger RNA (mRNA), ribosomal RNA (rRNA), transfer RNA (tRNA) as well as small RNA species, such as microRNA (mi RNA), that have regulatory function. “Small RNA species” have a specific meaning and refer to untranslated RNAs or non-coding RNAs with housekeeping or regulatory roles. “Small RNA species” are not rRNA or tRNA.

As used herein, the term “RNase inhibitor” refers to a chemical or other agent having the ability to interfere with the action of RNase enzymes, such as the endogenous RNases produced by many cells. An RNase is a ribonuclease, an enzyme that catalyzes the cleavage between nucleotides in RNA.

As used herein, the term “reverse transcription followed by polymerase chain reaction”, or “RT-PCR”, refers to a technique for synthesizing and amplifying a DNA molecule with a sequence that is a copy of an RNA sequence. RT-PCR is useful for detecting RNA species such as in quantitative analysis of gene expression, as well as for signal amplification in in-situ hybridizations. The technique consists of two parts: synthesis of cDNA from RNA by reverse transcription (RT), and amplification of a specific cDNA by polymerase chain reaction (PCR). Reverse transcriptase is an RNA dependent DNA polymerase that catalyses the polymerization of nucleotides using template RNA or the RNA molecule in an RNA:DNA hybrid.

As used herein, the term “primer” refers to an oligonucleotide, synthetic or naturally occurring, having a 3′-OH, which is capable of acting as a point of initiation of nucleic acid synthesis or replication along a template strand when placed under conditions in which the synthesis of a complementary strand is catalyzed by a polymerase. Within the context of reverse transcription, primers are composed of nucleic acids and prime on RNA or DNA templates. Within the context of PCR, primers are composed of nucleic acids and prime on DNA templates.

As used herein, the term “RT-PCR composition” means a composition having some or optionally all of the elements required to perform a one-step RT-PCR reaction including, but not limited to: primers, a polymerase, dNTPs, MgCl₂, and appropriate buffers. Optionally these compositions may also include a reverse transcriptase, an RNase inhibitor, and other reagents that may aid in the performance of an RT-PCR reaction.

As used herein, the term “RT composition” means a composition having some or optionally all of the elements required to synthesize a DNA product from an RNA template, including but not limited to nucleic acid primer(s) complementary to the target RNA, dNTPs, and the appropriate buffers. Optionally these compositions may include a reverse transcriptase, and an RNase inhibitor.

As used herein, the term “PCR composition” means a composition having some or optionally all of the elements required to amplify a DNA template, including but not limited to nucleic acid primers, polymerases, dNTPs and appropriate buffers. Optionally these compositions may also include an RNase inhibitor and may contain detection dyes or probes.

As used herein, the term “thermocycling” refers to the entire pattern of changing temperature used during an RT-PCR or PCR reaction. This process is common and well known in the art. In general, PCR thermocycling includes an initial denaturing step at high temperature, followed by a repetitive series of temperature cycles designed to allow template denaturation, primer annealing, and extension of the annealed primers by the polymerase. Generally, the samples are heated initially for 2-5 minutes to denature the double stranded DNA. Then, in the beginning of each cycle, the samples are denatured for 0.1 to 60 seconds, depending on the samples and the type of instrument used. After denaturing, the primers are allowed to anneal to the target DNA at a lower temperature, from about 45° C. to about 70° C. for about 20 to 60 sec. Under certain condition, the primer(s) may optionally be extended by the polymerase at a temperature ranging from about 65° C. to about 75° C. The amount of time used for extension will depend on the size of the amplicon and the type of enzymes used for amplification. The current rule of thumb is 1 min for 1 kb of DNA to be amplified. In addition, the annealing can be combined with the extension step, resulting in a two-step cycling. Thermocycling may include additional temperature shifts used in RT-PCR and PCR assays.

As used herein, the term “RT reaction mixture” means a mixture comprising all the elements required to perform reverse transcription and all or a portion of a cellular extract produced using an extraction medium as described herein. Typically, the RT reaction mixture is formed by contacting an RT composition with a cellular extract. In instances where all reagents needed for reverse transcription are not provided by the RT composition, the RT reaction mixture may be formed by contacting the cell extract with an RT composition and with any additional reagents required to perform reverse transcription.

As used herein, the term “C.T,” “Cycle Threshold,” “Threshold cycle,” or “Ct” refers to the cycle during thermocycling in which the increase in fluorescence due to product formation reaches a significant level above background signal.

As used herein, the term “RT-PCR reaction mixture” means a mixture comprising all the reagents required to perform one-step RT-PCR and all or a portion of a cellular extract produced using an extraction medium as described herein. Typically, the RT-PCR reaction mixture is formed by contacting an RT-PCR composition with a cellular extract. In instances where all reagents needed for one-step RT-PCR are not provided by the RT-PCR composition, the RT-PCR reaction mixture may be formed by contacting the cellular extract with an RT-PCR composition and with any additional reagents required to perform one-step RT-PCR.

As used herein, the term “PCR reaction mixture” means a mixture comprising all the elements required to perform PCR and all or a portion of an RT reaction product. Typically, the PCR reaction mixture is formed by contacting a PCR composition with an RT reaction product. In instances where all reagents needed for PCR are not provided by the PCR composition, the PCR reaction mixture may be formed by contacting an RT reaction product with a PCR composition and with any additional reagents required to perform PCR.

As used herein, the term “RT reaction product” means the product produced from the RT reaction mixture as a result of a reverse transcription reaction.

As used herein, the term “oligonucleotide” means a polymer of at least two nucleotides joined together by phosphodiester bonds and may consist of either ribonucleotides or deoxyribonucleotides. The term “oligonucleotice” is also meant to include modified oligonucleotides, such as oligonucleotides that have been chemically altered.

As used herein, the term “nucleic acid” generally refers to a molecule or strand of DNA, RNA, or derivatives or analogs thereof including one or more nucleobases. Nucleobases include purine or pyrimidine bases typically found in DNA or RNA (e.g., adenine, guanine, thymine, cytosine, and/or uracil). Nucleic acids may be single-stranded molecules, or they may be double-, triple-, or quadruple-stranded molecules that may include one or more complementary strands of a particular molecule. The term “nucleic acid” is also meant to include modified hucleic acids, such as nucleic acids that have been chemically altered.

Having described the invention in detail, it will be apparent that modifications and variations are possible without departing from the scope of the invention defined in the appended claims.

The following non-limiting examples are provided to further illustrate the present invention.

### EXAMPLE 1

### Effectiveness of Various Extraction Solutions for Feleasing and Protecting mRNA for Direct use in qRT-PCR

In this example, an extraction medium of the present invention was compared to commercially available extraction compositions, and to purified RNA for effectiveness at releasing and protecting mRNA from cells for use in quantitative RT-PCR (qRT-PCR) reactions.

### Materials and Methods

Unless otherwise noted, all materials were purchased from Sigma-Aldrich, Co., St. Louis, Mo.

Preparation of cells. HEK293 cells were grown in T75 cm² flasks using standard cell culture techniques. The cells were trypsinized, washed with phosphate buffered saline (PBS), and seeded in media at a concentration of 20,000 cells/well in a 96-well tissue culture treated microtiter plate. The cells were allowed to attach to the wells overnight at 37° C. with 5% CO₂ prior to aspirating media. Cell monolayers were then washed with 200 μl of PBS (Sigma catalog # D8662) pre-chilled to 2-8° C.

RNA extraction. RNA was extracted from the monolayers using Sigma's GenElute™ Mammalian Total RNA Miniprep Kit (Sigma catalog # RTN70) (“RTN Neat” or extract “G”) or Ambion's Cells-to-Signal Kit (Ambion, Inc., Austin Tex., catalog # 1726) (“Cells-to-Signal” or extract “A”), per manufacturer's recommendations. Dilutions of the RTN Neat RNA were also prepared by performing a 1:10 dilution of RTN Neat with water (“RTN 1:10” or extract “H”).

Crude extracts were also prepared by applying 100 μl of an extraction solution that was supplemented with 1.6 units/μl RNase inhibitor to the monolayers, incubating for 10 minutes at ambient temperature, and mixing until homogenous by pipetting up and down. The composition of each extraction solution, designated by a letter, is listed in Table 1. Each RNA extract was prepared in triplicate.

TABLE 1

| Extraction | |
|---|---|
| Solution | Formulation |
| B | 0.5% CHAPS, 150 mM NaCl, 25 mM bicine buffer, pH 7.6 |
| C | 1.5 mM MgCl₂, 0.42 M NaCl, 0.2 mM EDTA, 25% glycerol, |
| | and 20 mM HEPES, pH 7.9, 0.6% Igepal ® CA-630 |
| D | 1% triton X-100, 150 mM NaCl, 50 mM bicine buffer, pH 7.6 |
| E | 1% triton X-100, 300 mM NaCl, 5% gycerol, 100 mM |
| | tris-HCl, pH 8 |
| F | 150 mM NaCl, 1.0% Igepal ® CA-630, 0.5% sodium |
| | deoxycholate, 0.1% SDS, 50 mM Tris, pH 8.0. |

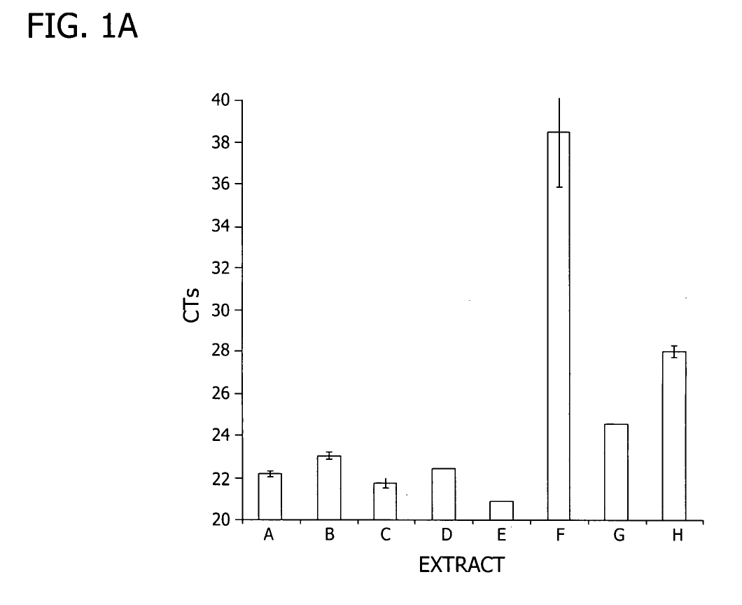

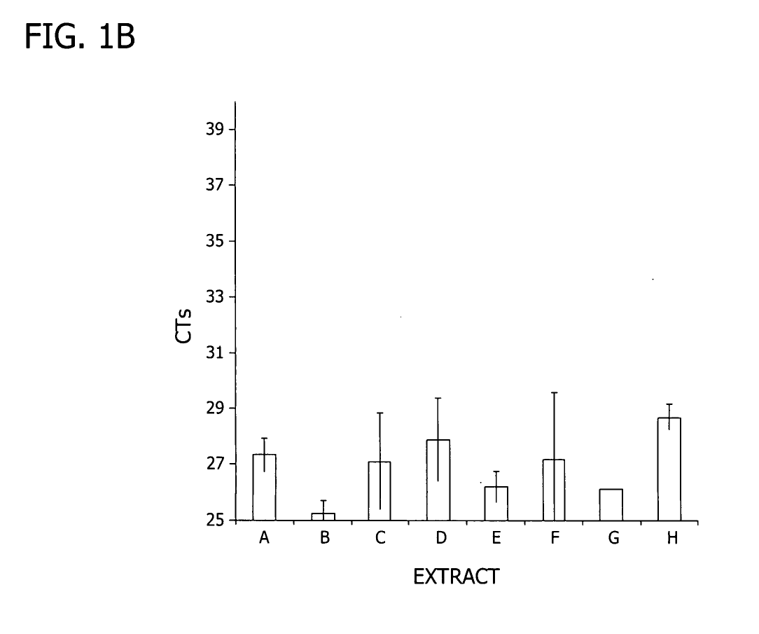

One-step qRT-PCR. 5 μl of the resulting RNA extracts were used in multiplexed one-step qRT-PCR reactions. The extracts were each combined with 2.5 μl 20X TaqMan primer & probe mix for glyceraldehyde-3-phosphate dehydrogenase (GAPDH) (Applied Biosystems, catalog # 4310884E), 2.5 μl 20X TaqMan primer & probe mix for phosphoglycerate kinase 1 (PGK1) (Applied Biosystems, catalog # 4333765F), 0.4 units/μl RNase inhibitor (Sigma catalog # R2520), 1X reference dye (Sigma catalog # R4526), 1 μl of MMLV-RT (25 U/μl), and 1X Probe Based qRT-PCR ReadyMix (Sigma catalog # P5871, from a kit, Sigma catalog # QR0200) to a final volume of 50 μl. A control without RNA was also prepared by combining these reagents with water to a final volume of 50 μl (“No Template”). Additionally, a control without a reverse transcriptase was prepared by combining RNA produced using Sigma's GenElute™ Mammalian Total RNA Miniprep Kit (Sigma catalog # RTN70) with these reagents minus MMLV-RT (“No RT Control”). The reaction mixtures were placed in a Stratagene Mx3000p real-time thermal cycler and qRT-PCR was carried out under the following conditions: The reaction mixtures were incubated at 45° C. for 45 minutes, then the temperature was raised to 94° C. for 3 minutes, followed by 40 cycles of 94° C. for 15 seconds and 60° C. for 1 minute, during which data was collected. The results are shown in FIG. 1A (GAPDH Ct values), FIG. 1B (PGK1 Ct values), and Table 2.

TABLE 2

| | | | | Standard |
|---|---|---|---|---|
| Extraction | Average of | Standard Deviation of | Average of | Deviation of |
| Solution | GAPDH CTs | GAPDH CTs | PGK1 CTs | PGK1 CTs |
| A (Cells-to-Signal) | 24.5 | 0.23 | 30.76 | 0.34 |
| B | 25.2 | 0.33 | 25.74 | 0.22 |
| C | 23.1 | 0.63 | 26.81 | 0.22 |
| D | 24.3 | 0.25 | 29.95 | 0.33 |
| E | 23.1 | 0.42 | 27.00 | 2.61 |
| F | 40.0 | 0.00 | 26.61 | 2.71 |
| G (RTN Neat)** | 24.6 | | 26.11 | |
| H (RTN 1:10) | 28.0 | 0.27 | 28.68 | 0.48 |
| No RT Control | 40.0 | 0.00 | 40.00 | 0.00 |
| (RTN Neat)* | | | | |
| No Template | 40.0 | 0.00 | 40.00 | 0.00 |
| (water)*** | | | | |

*MMLV-RT was not added to this reaction.
**Only 1 reaction was perform, so no standard deviation.
***No RNA extract was used in this reaction.

### Results and Discussion

GAPDH and PGK1 were detected in all reactions containing HEK293 cells. A comparison of CT values derived from crude extracts prepared with solutions B-E to a commercially available RNA purification product (e.g., Sigma's GenElute Mammalian Total RNA Miniprep Kit, Sigma catalog # RTN70) show that extracts prepared with extraction solutions B-E perform similarly to purified RNA (see Table 2). When the above-described procedure was repeated with extraction for 10 minutes at 65° C. instead of 10 minutes at ambient temperature, RNA release was not significantly improved (data not shown).

The reactions lacking MMLV-RT did not generate a GAPDH or PGK1 signal, demonstrating that the GAPDH and PGK1 primers do not amplify DNA. Therefore, the lack of signal in reactions lacking MMLV-RT indicates that signals produced by the qRT-PCR are attributed solely to the amplification of RNA.

### EXAMPLE 2

### Extraction of RNA from Cells with and without an RNase Inhibitor

In this example, RNA was extracted from cells using extraction solutions that did not contain an RNase inhibitor.

Preparation of cells. Hek293 cells were grown in T75 cm² flasks using standard cell culture techniques. The cells were trypsinized, washed with PBS, and seeded in media at a concentration of 20,000 cells/well in 96-well tissue culture treated microtiter plate. The cells were allowed to attach to the wells overnight at 37° C. with 5% CO₂ prior to aspirating media. Cell monolayers were then washed with 200 μl of PBS (Sigma catalog # D8662) pre-chilled to 2-8° C.

RNA extraction. RNA was extracted from the monolayers using Ambion's Cells-to-Signal Kit (Ambion, Inc., Austin Tex., catalog # 1726) per manufacturer's recommendations (“Cells-to-Signal” or extract “A”). Crude extracts were also prepared using extraction solutions B and E (components listed in Table 1). The crude extracts were generally prepared as described in Example 1, except that the extracts were prepared using either the extraction solution and 1.6 units/μl of RNase inhibitor or the extraction solution and no RNase inhibitor. The ambient temperature incubation times for each extract were taken at 0, 10, 20, and 60 minutes.

Controls were also prepared by extracting RNA from the monolayers using Sigma's GenElute™ Mammalian Total RNA Miniprep Kit (Sigma catalog # RTN70) per manufacturer instructions (“RTN-Neat”). Dilutions of the RTN Neat was also prepared by performing a 1:10 dilution of RTN Neat with water (“RTN-1:10”).

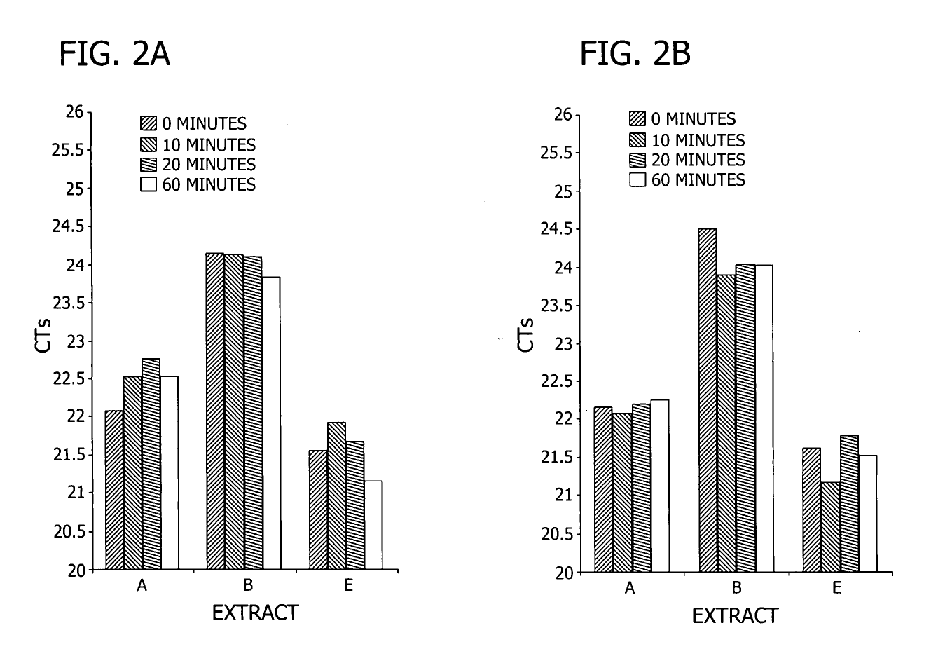

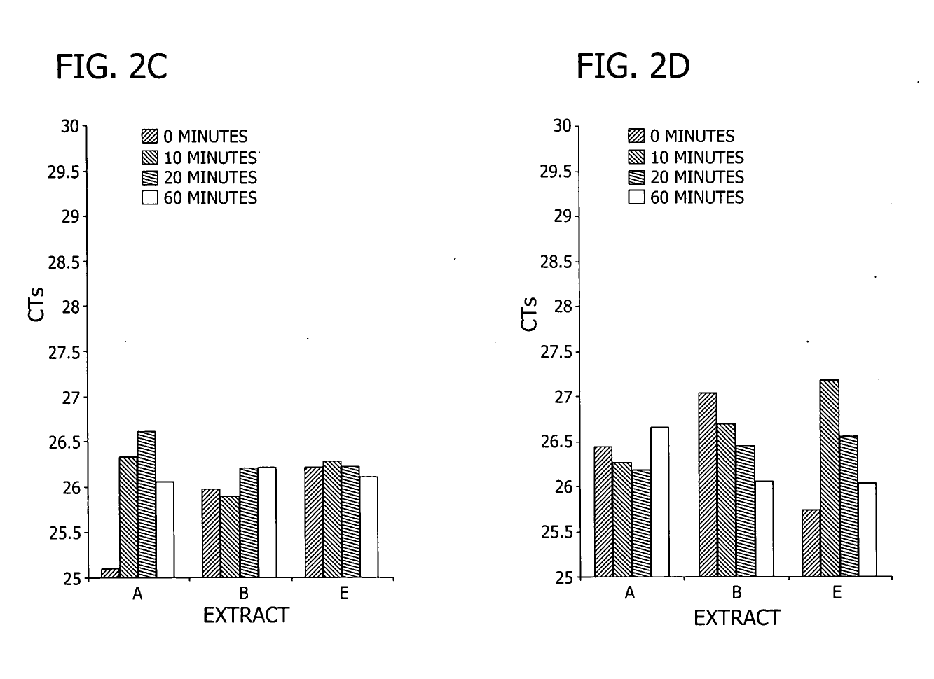

One-step qRT-PCR. Extracted RNA was amplified by qRT-PCR in an Applied Biosystems 7700 real time thermal cycler with GAPDH (Applied Biosystems catalog # 4310884E) or PGK1 (Applied Biosystems catalog # 4333765F) primers as described in Example 1. A control without RNA was also prepared as described in Example 1 (“No template control”). Additionally, a control without a reverse transcriptase was prepared using the RTN Neat RNA as described in Example 1 (“No RT control (RTN Neat)”). The results are shown in FIG. 2A (GAPDH Ct values for extracts prepared without RNase inhibitor), FIG. 2B (GAPDH Ct values for extracts prepared with RNase inhibitor), FIG. 2C (PGK1 Ct values for extracts prepared without RNase inhibitor), and FIG. 2D (PGK1 Ct values for extracts prepared with RNase inhibitor).

### Results and Discussion

No significant difference was seen in GAPDH or PGK1 mRNA levels (±˜1 CT) in crude extracts with and without RNase inhibitor or when extraction solution incubation times were varied from 0 minutes to 1 hour. Thus, supplementing extraction solution with RNase inhibitor or extending incubation times beyond 0 minutes provides no additional advantage.

RNA obtained using Sigma's GenElute™ Mammalian Total RNA Miniprep Kit (“RTN-Neat” or “RTN-1:10”) resulted in GAPDH CT values of 24.48 (RTN-Neat) and 27.92 (average of three samples of RTN-1:10), and PGK1 CT values of 26.86 (RTN-Neat) and 29.89 (average of three samples of RTN-1:10). A comparison of CT values for crude extracts prepared with extraction solution E (with or without an RNase inhibitor) to RNA obtained using the GenElute™ Mammalian Total RNA Miniprep Kit shows that extracts prepared with extraction solution E perform similarly to or better than the purified RNA.

### EXAMPLE 3

### Reproducibility of RNA Extraction

In this example, RNA extracts were prepared from multiple 96-well cultures for direct use in qRT-PCR to evaluate extraction-to-extraction variation.

### Materials and Methods

Preparation of cells. THP1 cells were grown using standard cell culture techniques, harvested by centrifugation at 800×g, washed with PBS, and seeded at a concentration of 50,000 cells/well in 96-well tissue culture treated microtiter plates. Cell pellets were formed by spinning the plates at 1,200×g for 5 minutes and then aspirating the supernatant.

RNA extraction. Crude extracts were prepared by applying 100 μl of either extraction solution E (i.e., 1% triton X-100, 300 mM NaCl, 5% glycerol, and 100 mM tris-HCl, pH 8) or extraction solution E without NaCl (i.e., 1% triton X-100, 5% glycerol, and 100 mM tris-HCl, pH 8) to the pelleted THP1 cells, incubating for 10 minutes at ambient temperature, and mixing until homogenous by pipetting up and down. Twenty-two replicates were prepared with each extraction solution (i.e., E or E without NaCl).

One-step qRT-PCR. 5 μl of the resulting RNA extracts were used in multiplexed one-step qRT-PCR reactions. The extracts were each combined with 1.25 μl of 20X TaqMan primer and probe mix for PGK1 (Applied Biosystems, catalog # 4333765F), 1X reference dye (Sigma catalog # R4526), 1 μl of MMLV-RT (25 units/μl), and 1X Probe Based qRT-PCR ReadyMix (Sigma catalog # P5871, from Sigma kit, catalog # QR0200) to a final volume of 25 μl. Reactions were qRT-PCR amplified in a Stratagene Mx3000p real-time thermal cycler as described in Example 1.

### Results and Discussion

The average PGK1 Ct value for extracts made with extraction solution E was 29.5, and the standard deviation was 0.8. Extracts made with extraction solution E without NaCl produced an average PGK1 Ct of 28.3, and a standard deviation of 0.4. These results show that extraction-to-extraction variation is negligible when using crude extracts for relative quantitation of mRNA. The higher Ct value for extracts made with extraction solution E than for extracts made with extraction solution E without NaCl may be the result of high salt concentration interfering with the PCR reaction when the NaCl in extraction solution E is added to the salt in 1X Probe Based qRT-PCR ReadyMix used in the PCR reaction. This explanation is tested in Example 4.

Similar results were obtained with GAPDH primers. The above procedure was repeated, except 20X TaqMan primer and probe mix for GAPDH (Applied Biosystems, catalog # 4310884E) was used instead of the PGK1 mix, and only an extract using extraction solution E without NaCl was prepared. Twenty-one replicates were prepared with the extraction solution. Extracts made with extraction solution E without NaCl produced an average GAPDH Ct value of 18.3, and a standard deviation of 1.0, again showing good extraction-to-extraction reproducibility.

### EXAMPLE 4

### Removal of KCl from RT-PCR Composition

An extraction medium comprising 1% triton X-100, 300 mM NaCl, 5% glycerol, and 100 mM tris-HCl, pH 8 adds 60 mM salt to the 50 mM KCl typically contained in a RT-PCR reaction mixture, when 5 μl crude extract is used for a template. In this example, the performance of one-step qRT-PCR with this concentration of salt (˜110 mM) was compared directly to one-step qRT-PCR with a reduced salt concentration (60 mM NaCl) by removing KCl from the RT-PCR composition to compensate for NaCl in the extraction medium.

### Materials and Methods

Cell preparation. Panc1 cells were grown in T75 cm² flasks using standard cell culture techniques. The cells were trypsinized, washed with PBS, and seeded in media at a concentration of 50,000 cells/well, in a 96-well tissue culture treated microtiter plate. The cells were allowed to attach to the wells overnight at 37° C. with 5% CO₂ prior to aspirating media. Cell monolayers were then washed with PBS, as described in Example 1.

RNA extraction. Crude extracts were prepared by applying 100 μl of extraction solution E (components listed in Table 1) to the Panc1 monolayers, incubating for 10 minutes at ambient temperature, and mixing until homogenous by pipetting up and down.

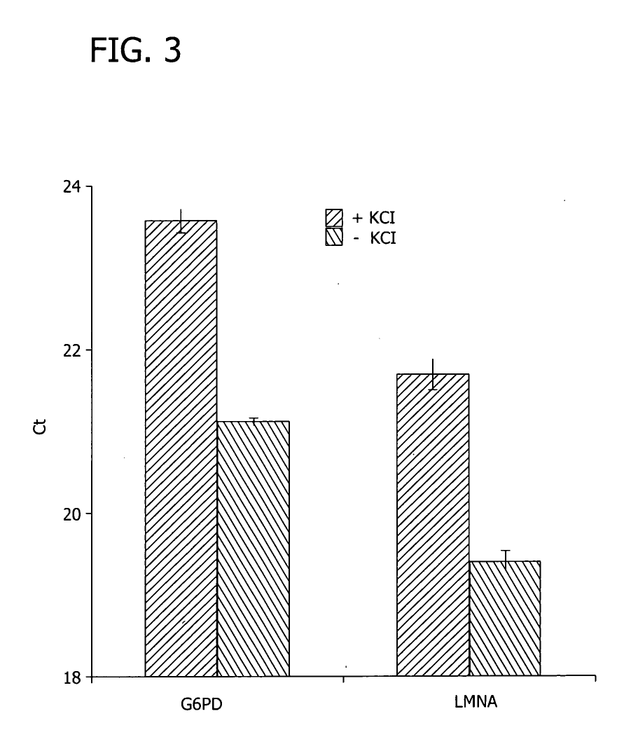

One-step qRT-PCR. One-step qRT-PCR was performed in a total reaction volume of 25 μl by combining 5 μl crude extract, 50 mM KCl, 15% glycerol, 3 mM MgCl₂, 0.2 mM each dNTP, 1 unit/μl MMLV-RT, 0.4 units/μl RNase inhibitor, 0.05% BSA, 0.2 μM forward and 0.2 μM reverse primer, 0.1 μM dual-labeled fluorogenic probe, and 10 mM tris, pH 8.3. One-step qRT-PCR was also performed under these same conditions, except the addition of 50 mM KCl was omitted. The primers and dual-labeled fluorogenic probes targeted either glucose-6-phosphate dehydrogenase (G6PD) or Lamin A (LMNA). For reactions targeting G6PD, the G6PD forward primer was 5′-CCTGACCTACGGCAACAGAT (SEQ. ID. NO. 1), the G6PD reverse primer was 5′-CTCTTCATCAGCTCGTCTGC (SEQ. ID. NO. 2), and the G6PD probe was 5′-TCTGCGGGAGCCAGATGCACT (SEQ. ID. NO. 3), the G6PD probe having a FAM dye at the 5′ end and a DBH1 quencher at the 3′ end. For reactions targeting LMNA, the LMNA forward primer was 5′-GATGATCCCTTGCTGACTTACC (SEQ. ID. NO. 4), the LMNA reverse primer was 5′-GTCGTCCTCAACCACAGTCAC (SEQ. ID. NO. 5), and the LMNA probe was 5′-CCACTGGGGMGAAGTGGCCATGCG (SEQ. ID. NO. 6), the LMNA probe having a JOE dye at the 5′ end and a DBH1 quencher at the 3′ end. The reaction mixtures were qRT-PCR amplified in a Stratagene Mx3000p real-time thermal cycler as outlined in Example 5, discussed below. The results are shown in FIG. 3.

### Results and Discussion

G6PD and LMNA qRT-PCR results were better (i.e., lower Ct) for the reaction mixtures without 50 mM KCl than for the standard reaction mixture that contains 50 mM KCl. The crude extracts contained 300 mM NaCl, and since 5 μl of the crude extract was used in the total reaction volume of 25 μl, the final NaCl concentration was 60 mM. 60 mM NaCl in addition to the 50 mM KCl used in a typical PCR reaction had inhibitory effects on RT and/or PCR. This inhibition is easily alleviated by removing KCl from the RT-PCR composition, thereby giving rise to reaction mixtures containing 60 mM NaCl with no KCl.

### EXAMPLE 5

### Simultaneous RNA Extraction and First Strand cDNA Synthesis

In this example, RNA was extracted from cells and cDNA was synthesized in the extraction solution. The resulting product was used directly in qPCR.

### Materials and Methods

Preparation of cells. Panc1 cells were grown using standard cell culture techniques. The cells were seeded at concentrations of 50,000, 30,000, 10,000, 1,000, or 100 cells/well, in 96-well tissue culture treated microtiter plate, incubated overnight, and washed with PBS as described in Example 1.

RNA extraction and cDNA synthesis. Crude extracts were prepared by applying 100, 75, 50, or 25 μl of an extraction solution comprising 250 mM KCl, 1% triton X-100, 5% glycerol, 3 mM MgCl₂, 0.2 mM of each dNTP, 1 unit/μl MMLV-RT, 0.4 units/μl RNase inhibitor, 5 mM dithiothreitol (DTT), 3.5 μM oligo dT, and 100 mM tris, pH 8, to the Panc1 monolayers, and mixing by pipetting up and down. The liquid from each well was transferred to a 96-well PCR plate, and incubated for 15 minutes at 42° C. in a PE9700 thermal cycler to synthesize cDNA.

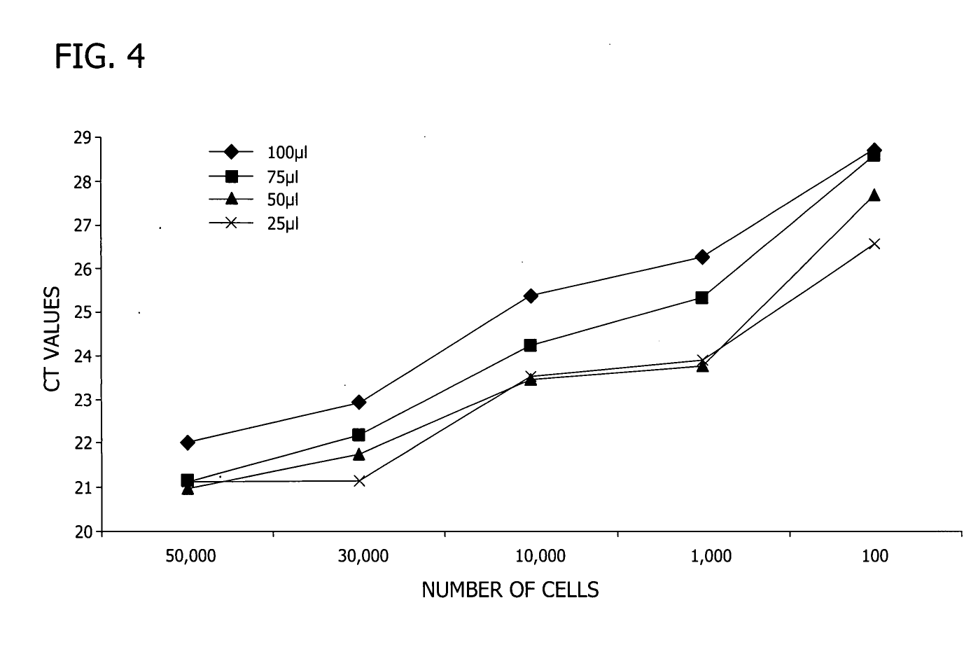

qPCR. 5 μl of the resulting product comprising cDNA was used directly in qPCR. The cDNA was combinined with 1.25 μl of 20X TaqMan primer and probe mix for PGK1 (Applied Biosystems, catalog # 4333765F), 1X reference dye (Sigma catalog # R4526), 3 mM MgCl₂, 0.2 mM of each dNTP, 0.4 unit/μl RNase inhibitor, 0.025 ppm kathon, 0.025% BSA, and 10 mM tris-HCl, pH 8.3 to a final volume of 25 μl. The reaction mixtures were amplified in a Stratagene Mx3000p real-time thermal cycler and qPCR was carried out under the following conditions: The reaction mixtures were incubated at 94° C. for 3 minutes, followed by 45 cycles of 94° C. for 15 seconds and 60° C. for 1 minute, during which time data was collected. The results are shown in FIG. 4.

### Results and Discussion

MMLV-RT was capable of polymerizing first strand cDNA synthesis under conditions that release mRNA from cells.

### EXAMPLE 6

### Comparison of Two-Step RT-PCR with One-Step RT-PCR

In this example, the sensitivity of two-step qRT-PCR and one-step qRT-PCR were compared.

### Materials and Methods

Preparation of cells. Hela cells were propagated using standard cell culture techniques. The cells were trypsinized, washed with PBS, and seeded in media at a concentration of 5,000 cells/well in a 96-well tissue culture treated microtiter plate. The cells were allowed to attach to the wells overnight at 37° C. with 5% CO₂.

A T25 flask was also seeded with the same culture of Hela cells and propagated. The cells were trypsinized, washed with PBS, and incubated in media at 37° C. with 5% CO₂ until optimal confluency was achieved. Cells were harvested at a concentration of 5000 cells/μl. These cells were used to prepare purified RNA for positive controls (RTN), described below.

To each well in the plate containing the Hela cells was added a 10-fold diluted siRNA (obtained from Dharmacon, Lafayette, Colo.) that targets either IRAK1, IRAK2, CHUK, MAP3K2, MAPK8, IKBKB, TNF, or IL-1b, all of which are part of the Nuclear Factor Kappa beta complex pathway, or siControl (Dharmacon, Lafayette, Colo.). DharmaFECT™ siRNA transfection reagent (Dharmacon, Lafayette, Colo.) was also added to the wells containing either the diluted siRNA or siControl, per manufacturer's instructions. The plate was incubated at 37° C. with 5% CO₂ for 24 hours, at which time the transfection reagent was removed by aspirating and replaced with media containing serum. The cells were incubated for an additional 24 hours, and the media was removed by aspiration. The Hela cells propagated in the T25 flask were not transfected with siRNA.

RNA extraction. Crude extracts were prepared from the Hela cells in the plate using extraction solution E (contents listed in Table 1) according to the following procedure: The cells were washed with cold PBS, which was then removed by aspiration. Extraction solution E was added to each well in the amount of 100 μl. The resulting extracts were used directly in one-step qRT-PCR.

Controls were also prepared by extracting RNA from the non-transfected Hela cells propagated in the T25 flask using Sigma's GenElute™ Mammalian Total RNA Miniprep Kit (Sigma catalog # RTN70) per manufacturer instructions (“RTN-Neat”). Three dilutions of the RTN Neat were also prepared. An 8-fold dilution of RTN Neat with water was performed to produce the first dilution (“RTN-D1”), followed by an 8-fold dilution of RTN-D1 to produce the second dilution (“RTN-D2”), and an 8-fold dilution of RTN-D2 to produce the third dilution (“RTN-D3”).

One-steD QRT-PCR. One-step qRT-PCR reactions were performed in a total reaction volume of 25 μl. 5 μl of each extract or RNA sample was combined with the following reagents: 1X reference dye (Sigma catalog # R4526), 10 mM Tris-HCl, pH 8.3, 3 mM MgCl2, 0.2 mM of each dNTP, 7.5% glycerol, 0.05 units/μl JumpStart™ Taq antibody (Sigma-Aldrich, Co., St. Louis, Mo.), 0.025% BSA, 0.4 units/μl RNase inhibitor, 0.025 ppm Kathon, 1.25 μl of the appropriate 20X specific gene expression assay (GEA) (Applied Biosystems, Inc., Foster City, Calif.), 1.25 μl 20X GAPDH GEA (Applied Biosystems, Inc., Foster City, Calif.), and 1 unit/μl MMLV-RT. The 20X specific GEAs were specific for one of the siRNA targets listed above.

Each GEA set contained the following reactions: (1) 3 samples containing the above listed qRT-PCR reagents and extracts of cells transfected with the specific siRNA; (2) 1 sample containing the above listed qRT-PCR reagents, minus MMLV-RT, and an extract of cells transfected with the specific siRNA; (3) 3 samples containing the above listed qRT-PCR reagents and extracts of cells transfected with siControl; (4) 1 sample containing the above listed qRT-PCR reagents, minus MMLV-RT, and extracts of cells transfected with siControl; (5) 3 samples containing the above listed qRT-PCR reagents and either RTN-D1, RTN-D2, or RTN-D3; and (6) a negative control comprising the above listed qRT-PCR reagents and water (“No template control”).

The reaction mixtures were placed in a Stratagene Mx3000p real-time thermal cycler and qRT-PCR was carried out under the following conditions: The reaction mixtures were incubated at 42° C. for 15 minutes, then the temperature was raised to 94° C. for 3 minutes, followed by 45 cycles of 94° C. for 15 seconds and 60° C. for 1 minute, during which data was collected. Each multiplexed qRT-PCR reaction contained a gene-specific GEA primer/probe set in addition to the GAPDH GEA primer/probe set, so that the level of target mRNA could be calculated by normalizing the signal generated from the specific GEA primer/probe set (which was FAM labeled) to the signal generated from the GAPDH GEA primer/probe set (which was HEX labeled).

Two-step qRT-PCR. The first reactions for two-step qRT-PCR were performed in a total reaction volume of 20 μl for each sample. 4 μl of each extract or RNA sample was combined with the following reagents: 0.5 mM of each dNTP, 5 μM random nonamers, 1 unit/μl RNase inhibitor, 1.25 units/μl MMLV-RT, 50 mM Tris-HCl, pH 8.3, 3 mM MgCl₂, and 5 mM DTT. Reactions were laid out in a 96-well PCR plate and incubated at room temperature for 15 minutes. The plates were then placed in a PE9700 thermal cycler at 42° C. for 30 minutes followed by 10 minutes at 94° C. Reverse transcription was also performed on samples under the same conditions without a reverse transcriptase (“No RT control” samples).

qPCR reactions were then performed on the resulting cDNA in a total reaction volume of 20 μl. 2 μl of each cDNA sample was combined with the following reagents: 1X reference dye (Sigma catalog # R4526), 1X probe-based qRT-PCR ReadyMix (Sigma Catalog # P5871), 1.25 μl of the appropriate 20X specific GEA (Applied Biosystems, Inc., Foster City, Calif.), and 1.25 μl 20X GAPDH GEA (Applied Biosystems, Inc., Foster City, Calif.). The 20X specific GEAs were specific for one of the siRNA targets listed above.

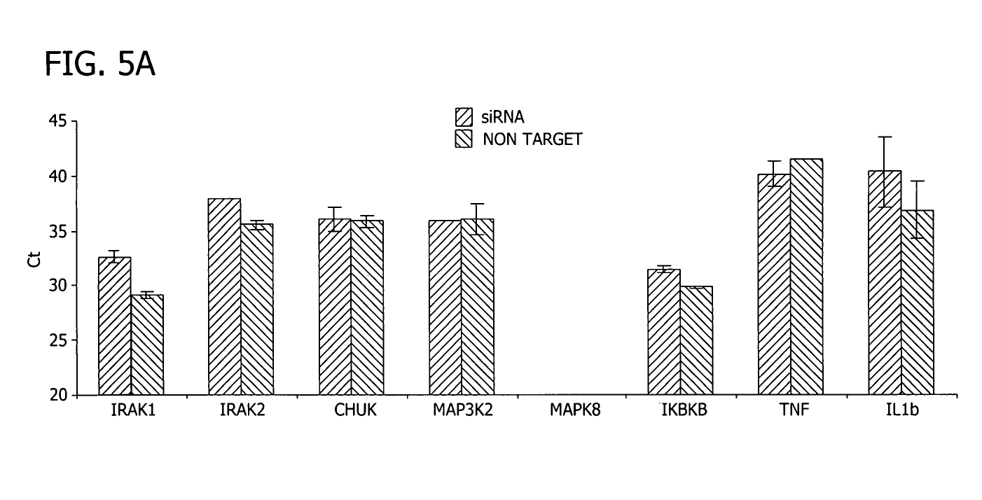

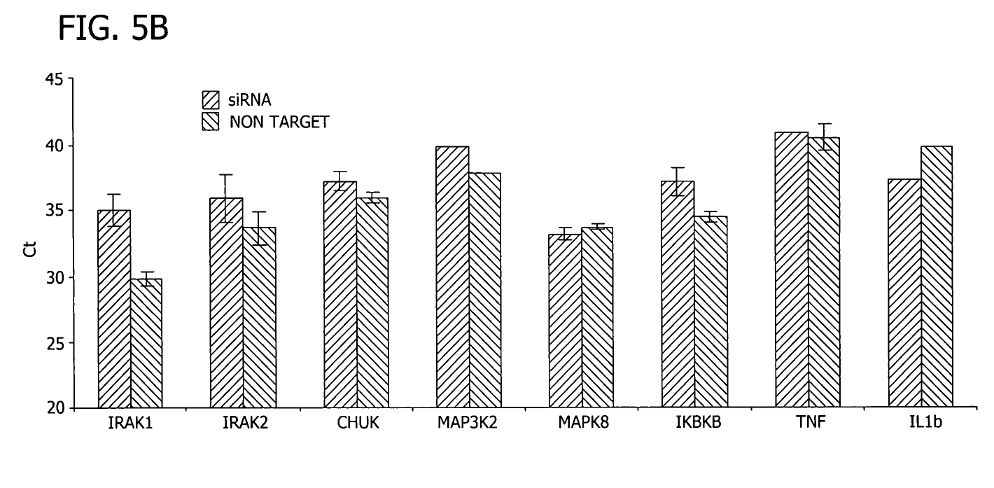

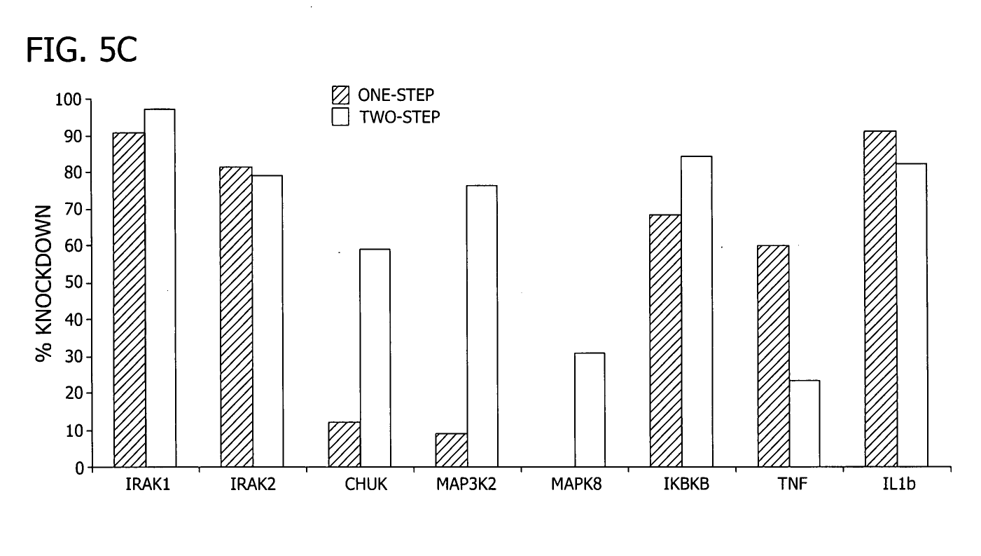

Each GEA set contained the following reactions: (1) 3 samples containing the above listed qPCR reagents and the reverse transcription product obtained from the extracts of cells transfected with the specific siRNA; (2) 1 sample containing the above listed qPCR reagents and the No RT Control product obtained from the extracts of cells transfected with the specific siRNA; (3) 3 samples containing the above listed qPCR reagents and the reverse transcription product obtained from the extracts transfected with siControl; (4) 1 sample containing the above listed qPCR reagents and the No RT Control product obtained from the extracts from cells transfected with siControl; (5) 3 samples containing the above listed qPCR reagents and the reverse transcription product obtained from the RTN-D1, RTN-D2, or RTN-D3 controls; and (6) a negative control comprising the above listed qPCR reagents and water. The reaction mixtures were placed in a Stratagene Mx3000p real-time thermal cycler and qPCR was carried out under the following conditions. The reactions were incubated at 94° C. for 3 minutes, followed by 45 cycles of 94° C. for 15 seconds and 60° C. for 1 minute, during which data was collected. Each multiplexed qRT-PCR reaction contained a specific GEA primer/probe set in addition to the GAPDH GEA primer/probe set, so that percent knockdown could be calculated by normalizing the signal generated from the specific GEA primer/probe set (which was FAM labeled) to the signal generated from the GAPDH GEA primer/probe set (which was HEX labeled). The results are shown in FIGS. 5A, 5B, and 5C.

### Results and Discussion

The sensitivity of two-step qRT-PCR was better than that of one-step qRT-PCR in this example. FIG. 5A shows the Ct values from the “target” (i.e., the siRNA transfected samples) vs. the “non-target” (i.e., the siControl samples) for the one-step method, and FIG. 5B shows the Ct values from the “target” vs. the “non-target” for the two-step method. FIG. 5C shows a comparison of the percent knockdown for the one-step and the two-step methods for each siRNA target. Percent knockdown calculates the extent to which each gene specific siRNA was able to reduce the level of its target mRNA compared to cells that received the non-target siRNA control. With two-step reactions, knockdown was more readily detected for 3 of the 8 targets and was more consistent with results for purified RNA (RTN).

### EXAMPLE 7

### Comparison of Two-Step qRT-PCR with Other Methods

In this example, the sensitivity of two-step qRT-PCR performed using RNA prepared from an extraction composition of the present invention was compared to a commercially available product designed to extract RNA and amplify it via qRT-PCR.

Preparation of cells. Hela cells were propagated using standard cell culture techniques. The cells were grown at 37° C. with 5% CO₂ until an optimal confluency was achieved, trypsinized, washed with PBS (Sigma Catalog # D8662), pre-chilled to 2-8° C., and diluted with PBS to a concentration of 5000 cells/μl. Aliquots containing 40,000 cells (8 μl) were transferred to separate tubes for extraction.

RNA extraction. Crude extracts were prepared from 40,000 cells using either extraction solution E (contents listed in Table 1) or Qiagen's FastLane cell cDNA kit (Qiagen, Inc., Valencia, Calif.). Purified RNA was prepared using Sigma's GenElute™ Mammalian Total RNA Miniprep Kit as a control (“RTN Control”). Extracts were prepared by adding 100 μl of extraction solution E (contents listed in Table 1) to each tube. Extracts prepared using Qiagen's FastLane cell cDNA kit, which requires two reagents used separately and an incubation at elevated temperature, was prepared per manufacturer's instructions. RNA prepared using Sigma's GenElute™ Mammalian Total RNA Miniprep Kit was also prepared per manufacturer's instructions.

Two-step QRT-PCR. Multiple reverse transcription reactions were prepared from each extract by combining 8 μl of extract with a reverse transcription reaction mixture in a total volume of 40 μl. RNA extracted with extraction solution E was combined with 0.5 mM of each dNTP, 5 μM random nonamers, 1 unit/μl RNase inhibitor, 1.25 units/μl MMLV-RT, 50 mM Tris-HCl, pH 8.3, 3 mM MgCl₂, and 5 mM DTT. Extracts prepared with Qiagen's FastLane cell cDNA kit were combined with the reverse transcription reaction mixture provided by the manufacturer. Positive control reactions were prepared for both RT reaction mixtures with the Sigma GenElute™ Mammalian Total RNA preparations. Reactions were incubated at room temperature for 15 minutes and then were placed in a PE9700 thermal cycler at 42° C. for 30 minutes followed by 10 minutes at 94° C. Each of the 40 μl reverse transcription reactions prepared using RNA extracted with Qiagen's FastLane cell cDNA kit was pooled prior to continuing with qPCR. Similarly, all the reverse transcription reactions prepared using extraction solution E were also pooled, as were the reverse transcription reactions prepared using Sigma's GenElute™ Mammalian Total RNA Miniprep Kit. Control reactions without reverse transcriptase were also prepared for RNA extracted by each extraction method by combining extracted RNA with the above listed reverse transcription reagents minus MMLV-RT (“No RT Control” samples).

qPCR reactions were then performed by combining 2 μl of each pooled reverse transcription product with the following reagents in a total reaction volume of 20 μl. cDNA prepared from extracts prepared using extraction solution E were combined with 1X reference dye (Sigma catalog # R4526), 1X probe-based qRT-PCR ReadyMix (Sigma catalog # P5871), 1.25 μl of the appropriate 20X specific gene expression assay (GEA) (Applied Biosystems, Inc., Foster City, Calif.), and 1.25 μl 20X GAPDH gene expression assay (GEA) (Applied Biosystems, Inc., Foster City, Calif.). cDNA prepared from extracts prepared using Qiagen's FastLane cell cDNA kit were added to the qPCR reagents from Qiagen's QuantiTect multiplex PCR kit along with the same GEA primers and probes as described above. The 20X specific gene expression assays were specific for one of the following mRNA targets: GAPDH, PGK1, TERT, IL8, GUK1, CKB, CHCHD2, RAC1, CDC42, PHKG1, GRB2, PKM2, MAP2K2, ACTB, B2M, GUSB, HPRT1, PPIA, RPLPO, TBP, TFRC, PKN1, VEGF, IRAK1, IRAK2, CX3CR1, LTA4H, CALM2, CXCL11, CXCL13, CXCL6, CHUK, MAP3K2, MAPK8, IKBKB, TNF, IL-1b, or LMNA.

In addition to the aforementioned gene specific primer/probe sets, Sybr green I based qRT-PCR was also performed by combining 2 μl of each pooled reverse transcription product with the following reagents in a total reaction volume of 20 μl: 1X reference dye (Sigma catalog # R4526), 1X SYBR-based qRT-PCR ReadyMix (Sigma catalog # P5191), and 0.5 μM of each SYBR-based primer was used. The SYBR-based primers were specific for one of the following mRNA targets: CREB1, FOS, GTF3A, HSF1, ELAVL1, MYC, SRF, XBP1, ATF-5, or SURF4-2.

Each GEA set or SYBR-based primer set contained the following reactions: (1) 4 replicates of the extract; (2) an RTN control; (3) 2 No RT Control samples; and (4) a negative control without RNA (comprising the qPCR reagents and water).

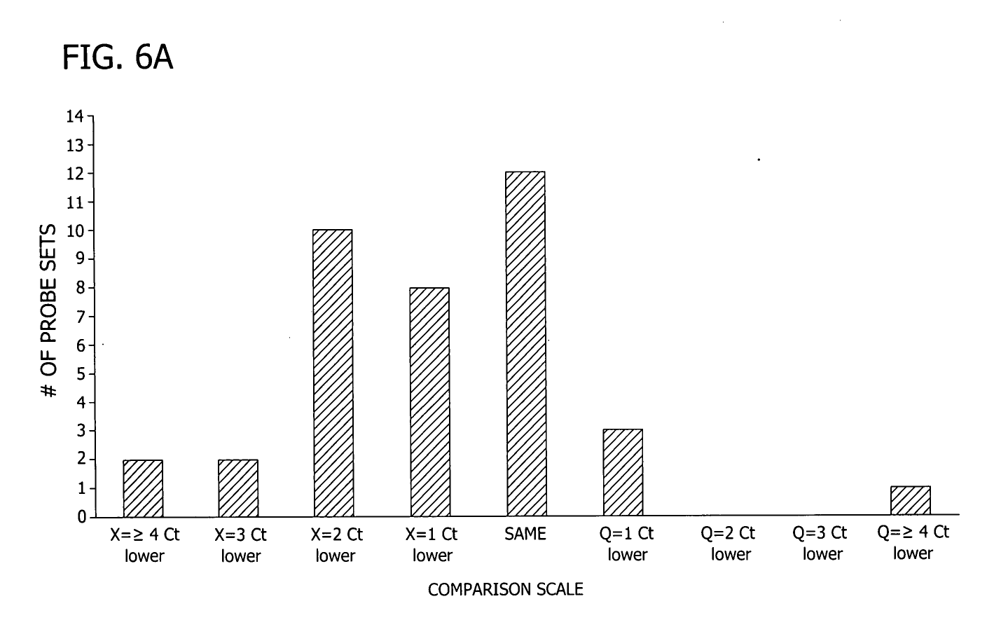

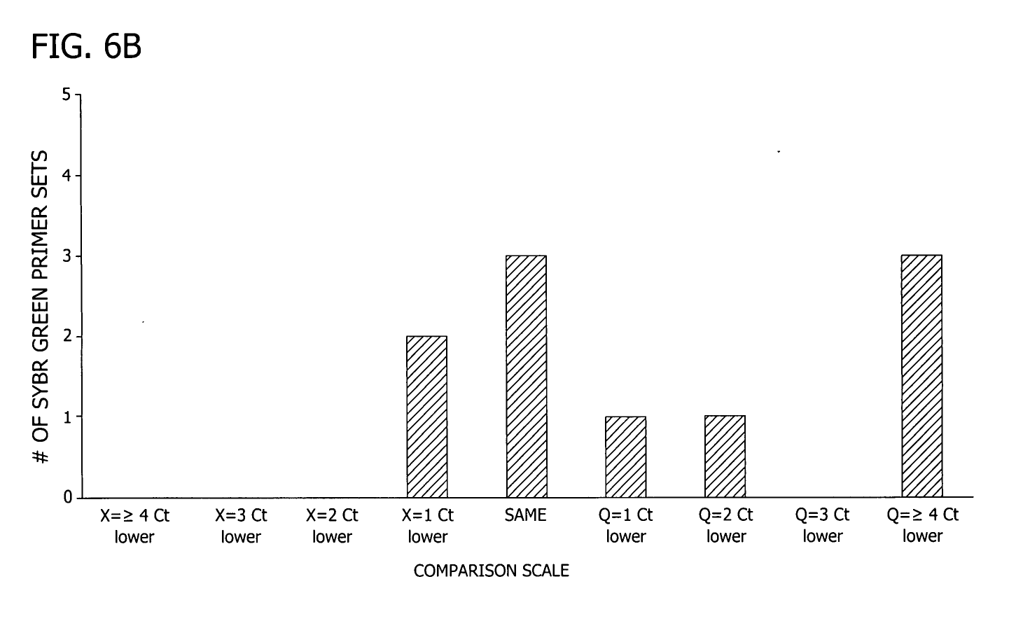

The reaction mixtures were placed in a Stratagene Mx 3000p thermal cycler and qPCR was carried out under the following conditions: The reactions were incubated at 94° C. for 3 minutes, followed by 45 cycles of 94° C. for 15 seconds and 60° C. for 1 minute, during which time data was collected. Reactions with Qiagen's Quantitect multiplex PCR kit were conducted according to the manufacturer's instructions. The results are illustrated in FIGS. 6A and 6B.

### Results and Discussion

FIG. 6A summarizes the qRT-PCR results for all 38 GEA sets. CT values (average for the 4 replicates) for the RNA prepared using the Qiagen kit are compared to the CT values (average for the 4 replicates) for extracts prepared using extraction solution E. CT values for 32% of the primer/probe sets (12 sets) were the same (within 0.5 cycles) for RNA produced with the Qiagen kit and extracts prepared using extraction solution E (“Same”). Extracts prepared using extraction solution E resulted in lower Ct values than RNA prepared using the Qiagen kit for 57% of the primer/probe sets (22 sets): 21% of these (8 sets) were lower by approximately 1CT (“X=1Ct lower”); 26% (10 sets) were lower by approximately 2 CTs (“X=2Ct lower”); 5% (2 sets) were lower by approximately 3 CTs (“X=3Ct lower”); and 5% (2 sets) were lower by approximately 4 or more CTs (“X=≧4Ct lower”). RNA prepared using the Qiagen kit produced lower Ct values than extracts prepared using extraction solution E for only 11% of the primer/probe sets (4 sets): 8% of these (3 sets) were lower by approximately 1 CT (“Q=1Ct lower”), and 3% (1 set) were lower by approximately 4 or more CTs (“Q=≧4Ct lower”).

FIG. 6B summarizes the results for all 10 siRNA targets for qRT-PCR performed using the SYBR based primers. CT values (average for the 4 replicates) for the RNA prepared using the Qiagen kit are compared to the CT values (average for the 4 replicates) for extracts prepared using extraction solution E. Ct values for 30% (3 primer sets) of the qRT-PCR reactions performed using the SYBR based primers were the same (within 0.5 cycles) for RNA produced with the Qiagen kit and the extracts prepared using extraction solution E (“Same”). Extracts prepared using extraction solution E resulted in lower Ct values than RNA prepared using the Qiagen kits for 20% of the reactions (2 primer sets); the results were lower by approximately 1CT (“X=1Ct lower”) for these reactions. RNA prepared using the Qiagen kit resulted in lower Ct values than extracts prepared using extraction solution E for 50% of the reactions (5 primer sets): 10% of these (1 set) were lower by approximately 1 CT (“Q=1Ct lower”); 10% (1 set) were lower by approximately 2 CTs (“Q=2Ct lower”); and 30% (3 sets) were lower by approximately 4 or more CTs (“Q=≧4Ct lower”).

Thus, the results showed equal to better Ct values with dual labeled fluorogenic probe detection when using an extraction composition of the present invention than obtained when using the Qiagen kit (see FIG. 6A). The results obtained using the Qiagen kit were slightly better when qRT-PCR was performed using the SYBR primer sets (see FIG. 6B). Thus, a one-step extraction with extraction solution E according to the current invention gives results in two-step RT-PCR that are comparable to or better than a commercially available product that uses a multi-step extraction procedure.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

What is claimed is:

1. A method for extracting RNA from cells, the method comprising combining a cell population with an extraction medium to form a cellular extract containing extracted RNA, a salt selected from the group consisting of monovalent salts, divalent salts, and combinations thereof, and a detergent selected from the group consisting of non-ionic detergents, zwitterionic detergents, and combinations thereof, the concentration of the detergent in the cellular extract being about 0.1% to about 10% by weight, and the concentration of the salt in the cellular extract being about 10 mM to about 5 M.

2. The method of claim 1 wherein the salt is a monovalent salt, and the concentration of monovalent salt in the cellular extract is from about 150 mM to about 5 M.

3. The method of claim 2 wherein the concentration of the monovalent salt in the cellular extract is about 300 mM.

4. The method of claim 1 wherein the cellular extract comprises about 1% by weight of the detergent.

5. The method of claim 1 wherein the monovalent salt is selected from the group consisting of sodium fluoride, sodium chloride, sodium bromide, sodium iodide, potassium fluoride, potassium chloride, potassium bromide, potassium iodide, and combinations thereof.

6. The method of claim 1 wherein the non-ionic detergent is selected from the group consisting of alkyl glucosides, alkyl maltosides, alkyl thioglucosides, glucamides, polyoxyethylenes, and combinations thereof.

7. The method of claim 1 wherein the extraction medium comprises 1% by weight of a non-ionic detergent, 300 mM of a monovalent salt, 100 mM of a buffer, and 5% by weight glycerol.

8. The method of claim 1 wherein the extraction medium further comprises a reagent selected from the group consisting of dNTPs, a reverse transcriptase, a primer, a buffer, and combinations thereof.

9. The method of claim 8 wherein the extraction medium further comprises a DNA polymerase.

10. A method for preparing cDNA, the method comprising:
combining a cell population with an extraction medium to form a cellular extract containing extracted RNA, a salt selected from the group consisting of monovalent salts, divalent salts, and combinations thereof, and a detergent selected from the group consisting of non-ionic detergents, zwitterionic detergents, and combinations thereof, the concentration of the detergent in the cellular extract being about 0.1% to about 10% by weight and the concentration of the salt in the cellular extract being about 10 mM to about 5 M;
combining the cellular extract with a reverse transcriptase to form a first reaction mixture, and
incubating the first reaction mixture to produce a cDNA.

11. The method of claim 10 wherein the first reaction mixture additionally comprises a DNA polymerase and the method additionally comprises amplifying the cDNA in the reaction mixture.

12. The method claim 11 wherein the first reaction mixture additionally comprises a detection probe or a dye that specifically binds to dsDNA.

13. The method of claim 10 further comprising combining the first reaction mixture containing cDNA with a DNA polymerase after incubating to form a second reaction mixture, and amplifying the cDNA in the second reaction mixture.

14. The method of claim 13 wherein the second reaction mixture additionally comprises a detection probe or a dye that specifically binds to dsDNA.

15. The method of claim 10 wherein the salt is a monovalent salt, and the concentration of monovalent salt in the cellular extract is from about 150 mM to about 5 M.

16. The method of claim 15 wherein the concentration of the monovalent salt in the cellular extract is about 300 mM.

17. The method of claim 10 wherein the cellular extract comprises about 1% by weight of the detergent.

18. The method of claim 10 wherein the monovalent salt is selected from the group consisting of sodium fluoride, sodium chloride, sodium bromide, sodium iodide, potassium fluoride, potassium chloride, potassium bromide, potassium iodide, and combinations thereof.

19. The method of claim 10 wherein the non-ionic detergent is selected from the group consisting of alkyl glucosides, alkyl maltosides, alkyl thioglucosides, glucamides, polyoxyethylenes, and combinations thereof.

20. The method of claim 10 wherein the extraction medium comprises 1% by weight of a non-ionic detergent, 300 mM of a monovalent salt, 100 mM of a buffer, and 5% by weight glycerol.
